(19) United States
(12) Patent Application Publication
(10) Pub. No.: US 2023/0302451 A1
(43) Pub. Date: Sep. 28, 2023

(54) Modular Microfluidic Methods and Devices

(71) Applicant: CAMBRIDGE ENTERPRISE LIMITED, Cambridge, Cambridgeshire (GB)

(72) Inventors: Florian Hollfelder, Cambridge Cambridgeshire (GB); Tomasz Kaminski, Cambridge Cambridgeshire (GB); Joachim De Jonghe, Cambridge Cambridgeshire (GB)

(21) Appl. No.: 18/020,392

(22) PCT Filed: Aug. 13, 2021
(86) PCT No.: PCT/GB2021/052111

§ 371 Date: Feb. 8, 2023

(30) Foreign Application Priority Data
Aug. 14, 2020 (GB) 2012744.5

(51) Int. Cl. B01L 3/00; C12N 15/10

(52) U.S. Cl. CPC B01L 3/502784; C12N 15/1065; B01L 2200/028; B01L 2200/16; B01L 2200/0652

(57) ABSTRACT

The invention relates to microfluidic methods; and modular devices and systems for implementing the methods. The invention also relates to uses of these devices and systems for biological analyses.

### FIELD OF THE INVENTION

The present invention relates to microfluidic methods; and modular devices and systems for implementing the methods. The invention also relates to uses of these devices and systems for biological analyses.

### BACKGROUND

Single cell multi-omics have the potential to provide a wealth of information. These methods can elucidate the relationships between gene regulation, transcription and translation by measuring different components from the same cell.

However, the current methods for analysing single cells are not optimised. Increased single-cell library quality in terms of capture and types of structures sequenced as well as higher throughput would provide more information to probe the underlying causes of a disease for example.

The present invention addresses this need.

### SUMMARY OF THE INVENTION

In a first aspect there is provided:
A bead-extracting microfluidics device for extracting a bead from a microfluidic droplet, the device comprising:
a supply channel, into which microfluidic droplets can be injected;
the supply channel comprising a bifurcated junction in fluid communication with a first exit channel and a second exit channel,
wherein the first exit channel is adapted to extract the bead from the droplet; and the second exit channel is adapted to extract the droplet, the second exit channel having a smaller entrance from the bifurcated junction into the second exit channel than the entrance into the first exit channel from the bifurcated junction; and wherein the second exit channel has a lower flow resistance than the first exit channel.

Also provided is:
A method of extracting a bead from a microfluidic droplet, the method comprising:
a) providing a microfluidic droplet, the droplet comprising a bead; and
b) extracting the bead from the microfluidic droplet to provide: i) the bead; and ii) a droplet which does not comprise the bead; and
A bead and one or more microfluidic droplets, wherein the bead and microfluidic droplet(s) comprise different analytes from a single cell, optionally wherein the analytes on the bead and the droplets comprise the same barcode.

In a second aspect there is provided:
A picoinjection microfluidic module for single cell analyses comprising:
   a) a supply channel, into which microfluidic droplets comprising cell or cell structure lysate can be injected wherein the supply channel comprises a droplet spacer; and
   b) a picoinjector, downstream from the droplet spacer, for injecting reagent into the droplets.

In a third aspect there is provided:
A modular microfluidic system, the system comprising:
a) a droplet generation microfluidic module, wherein the droplet generation microfluidics module is adapted for encapsulation of cells or cell structures, lysis reagent and beads in microfluidic droplets, the droplet generation microfluidic module comprising a droplet generation junction in fluid communication with one or more input channels, the one or more input channels for flowing cells, lysis reagent, partitioning fluid and beads into the droplet generation junction; and
b) the picoinjection microfluidic module.

In a fourth aspect, there is provided:
A method for single cell analyses, the method comprising:
a) encapsulating a cell or cell structure, lysis reagent and a bead in a microfluidic droplet;
b) incubating the droplets to release the content of the cell or cell structure; and
c) picoinjecting a reagent into the microfluidic droplet.

In a fifth aspect there is provided:
A method of single cell RNA analyses, the method comprising:
   a) encapsulating a cell or cell structure, and lysis reagent in a microfluidic droplet;
   b) incubating the droplet to release the RNA;
   c) combining the droplet with: i) a bead; and ii) reverse transcriptase reagent using droplet fusion.

In a sixth aspect, there is provided:
A modular microfluidics system for single cell RNA analyses, wherein the system comprises:
a) a droplet generation microfluidic module, wherein the droplet generation microfluidics module is adapted for encapsulation of cells or cell structures and lysis reagent in microfluidic droplets, the droplet generation microfluidic module comprising a droplet generation junction in fluid communication with one or more input channels, the one or more input channels for flowing cells, lysis reagent and partitioning fluid into the droplet generation junction;
and
b) a droplet fusion module, the droplet fusion module comprising a fusion junction adapted to fuse reverse transcriptase reagent and a bead with the microfluidic droplet, wherein the droplet fusion module comprises a droplet spacer upstream of the fusion junction.

In a seventh aspect, there is provided:
A method of separating nuclear RNA and cytoplasmic RNA for sequencing, the method comprising:
step a) encapsulating a cell and outer membrane lysis reagent in a microfluidic droplet, wherein the outer membrane lysis reagent lyses the outer membrane of the cell;
step b) adding nuclear membrane lysis reagent, wherein the nuclear membrane lysis reagent is for lysing the nuclear membrane; and splitting the droplet into a plurality of droplets to obtain: i) a droplet with nuclear lysate; and ii) a plurality of droplets with cytoplasmic lysate;
step c) adding reverse transcriptase reagent and sorting the droplets into a first droplet comprising nuclear lysate; and a second droplet set comprising cytoplasmic lysate; and
step d) performing reverse transcription of: i) the nuclear RNA and; ii) the cytoplasmic RNA, wherein the cDNA in the cDNA sequencing library comprises a barcode and optionally a UMI.

In an eighth aspect there is provided:
A method of sequencing RNA from a plurality of interacting cells, the method comprising:

step a) encapsulating a first cell and a second cell with a barcoded bead, the bead comprising a plurality of poly-T primers, each primer additionally comprising a first barcode and optionally a UMI;
step b) optionally adding a cell separation reagent;
step c) adding lysis reagent; removal of the bead from the droplet and splitting the droplet into a plurality of droplets to obtain a droplet with lysate from the first cell or cell structure;
and a droplet with lysate from the second cell or cell structure.

### DETAILED DESCRIPTION

References to the figures in the following description are by way of example only and are not intended to limit the definitions to the specific embodiments shown in the figures.

Bead Extracting Microfluidics Device

The bead extracting device removes a bead, for example a barcoded bead, from a microfluidic droplet. Analytes captured on the bead are removed along with the bead. Other analytes in the droplet can then be further analysed by adding different reagents to the droplet without the bead. Example workflows using this device are shown in FIG. 2. Examples of bead extractors are shown in FIG. 1 and how it works is described in Example 1. The features are further described below.

Microfluidic Droplet

By microfluidic droplet is meant a discrete volume of a first liquid in an immiscible second liquid.

Bead

A bead is a solid support which can capture biological analytes. The bead may be any shape. The bead may be a non-dissolvable bead or a dissolvable bead.

A bead is an efficient way to incorporate barcodes in a droplet where the bead is a support with barcodes attached. Where the bead is non-dissolvable, the barcodes are attached to the bead via a linker which may be cleavable to remove the barcode from the bead. The linker may be cleavable with UV. The bead may also comprise primers, for example when analysing RNA, poly-T primers which comprise the barcodes and optionally a UMI.

Where the bead is dissolvable, the microfluidic droplet is incubated at the recommended temperature and other conditions required (e.g. adding chemical compounds (by co-encapsulation, picoinjection or droplet fusion) for breaking of polymer bonds to dissolve the bead at any of the points in any of the methods.

The bead may be 10-100 μm in diameter, for example, 10, 20, 30, 40, 50, 60, 70, 80, 90 or 100 μm. For example, the bead may be approximately 60-65 μm in diameter.

A bead may also comprise capture agents for capturing an array of different analytes. A capture agent may be an antibody when the analyte is a protein, or the capture agent may be a complementary nucleic acid when the analyte is another nucleic acid.

Alternatively, the bead physically immobilises an analyte of interest within the bead.

The beads described here are also applicable to the modular microfluidic systems and methods described below.

Supply Channel

The supply channel is the inlet channel into which the microfluidic droplet is injected. This channel comprises a bifurcated junction. That is, the supply channel splits into two different channels: a first exit channel and a second exit channel. The first and second exit channels are in fluid communication with the supply channel meaning that liquid can move from the supply channel and into the first and second exit channels.

The dimensions of the channels may optionally be smaller than the diameter of a droplet. If the channel is wider and deeper (i.e. of a larger diameter) than a droplet then oil can flow around the droplets and a much larger volume of oil is needed for sufficient spacing between droplets. As a guide only, the supply channel in FIG. 1b is 120 μm wide and 80 μm deep.

First Exit Channel

The first exit channel is adapted to extract the bead (the bead is also surrounded by a very small amount of the droplet; the majority of the droplet is extracted via the second exit channel).

The angle between the supply channel and the first exit channel referred to is calculated with reference to the supply channel, the supply channel being at zero degrees.

This angle may be 90.1-179.9 degrees, for example, 90, 95, 100, 105, 110, 115, 120, 125, 130, 135, 140, 145, 150, 155, 160, 165, 170, 175 or 179.9 degrees. This angle is useful where the droplet and bead are divided passively, e.g. by the second exit channel having a larger diameter as shown in FIG. 1a and FIG. 1b.

Second Exit Channel

The second exit channel is adapted to extract the droplet. Both exit channels have an entrance into the channels from the bifurcated junction. This is shown in FIG. 1.

Smaller Entrance

The entrances are the start of the first and second channels, after the supply channel bifurcates. The second exit channel has a smaller entrance into the channel. Smaller refers to the cross-sectional area. The cross-sectional area, or hole into the channel, at the entrance, is reduced. This reduces access to the second exit channel which prevents the bead exiting via the second exit channel.

The cross-sectional area of the entrance of the second exit channel can be reduced in various ways all of which prevent an object the size of a bead from entering the second exit channel. For example, the overall diameter of the entrance may be reduced in size as shown in FIG. 1c. The diameter of the entrance portion of the first exit channel may be 2-500 microns. For example, 10, 20, 30, 40, 50, 60, 70, 80, 90, 100, 150, 200, 250, 300, 350, 400, 450 or 500 microns.

Or the entrance may have partial barriers erected in the entrance as shown in FIGS. 1a and 1b. This reduction in size may be as a result of barriers which protrude into the channel of the entrance of the second exit channel preventing a bead from exiting the bifurcated junction via the second exit channel.

The size of the entrance is suitably chosen to allow the bead to pass through the entrance and exit via the first exit channel. The diameter of the entrance of the second exit channel may be 10-90%, for example, 10, 20, 30, 40, 50, 60, 70, 80 or 90%, for example 40-60% of the diameter of the entrance of the first exit channel. The diameter of the entrance to the second exit channel is suitably chosen to prevent access of the bead to the second exit channel.

Diameter

Diameter is used as a measurement of the distance from one side of the channel to the other side of the channel; the length of the line bisecting the cross-sectional area of the channel. The channels may be tubes which are square, circular or rectangular in cross section. Diameter is used as the measurement for all of these possible geometries. For example, where the channel is square or rectangular in cross-section, the diameter refers to the width and depth of the channel.

Lower Flow Resistance

As well as preventing the bead from entering the second exit channel, the droplet (i.e. the droplet without the bead) is also encouraged to enter the second exit channel by reducing the flow resistance in the second exit channel after the entrance, compared with the flow resistance in the first exit channel. This can be done in a variety of ways, e.g. by increasing the diameter of the second exit channel downstream of the entrance portion as shown in FIGS. 1a and 1b (see (5)), and/or by increasing the flow into the first exit channel as shown in FIG. 1b (see (10)). The second exit channel may also or alternatively be connected to a source of negative pressure, for example an aspirating device or vacuum).

Droplet Splitter

The droplet splitter splits a droplet into a plurality of smaller droplets. Microfluidic arrangements which can perform this function include: a plurality of T-junctions; a single flow-focusing junction, or a step emulsification geometry. Splitting using flow focusing is shown in FIG. 21a.

Method

A method is provided for extracting a bead from a droplet, optionally using the bead extractor device described above.

The method may be used to analyse different analytes (molecules of interest, e.g. DNA, RNA, protein) in single cell lysate in a parallel manner. This can be done for example where the bead comprises a first analyte, which may be physically captured in the bead, or captured via a capture agent attached to the bead. Extraction of the bead from the droplet also extracts this first analyte. The remainder of the analytes in the cell lysate may be analysed using any of the modular microfluidic devices set out below. For example, one or more reagents may be injected into the droplet containing the remainder of the analytes in the cell lysate.

The bead may comprise a plurality of capture agents for capturing various analytes for example in a single cell lysate. The method may comprise binding a plurality of analytes on the different capture agents. One or more of the capture agents may be cleavable from the analyte. Upon cleavage, the first analyte attached to the first capture agent is released from the bead. The bead extractor is then used to extract the bead from the droplet, the bead comprising the other analytes captured on the capture agents whilst the first analyte and first capture agent are found in the droplet. The same process can be repeated to extract a second and third and so on analyte from the bead in a sequential manner.

The cleavable linker may comprise a site which is cleaved by UV. Alternatively, chemical cleavage may be used. Different types of cleavable linker allow sequential cleavage of different analytes captured by different capture agents present on the bead.

Products of Method

The bead extractor device and splitter produce a bead and one or more microfluidic droplets, the bead and droplet(s) both comprising analytes from the same cell, the analytes optionally having the same barcode. One or more of the analytes may be captured by capture agents which are molecules capable of binding analytes in a cell. For example, the bead may comprise a first capture agent for capturing a first analyte. In the droplet, there may be a second analyte, optionally captured by a second capture agent, the second capture agent cleaved from the bead prior to bead extraction.

By using the same barcode for different capture agents, different analytes from the same cell can be traced back to the cell. Other identifiers may also be used other than barcodes.

Use

The bead extractor device as described above may be used in various single cell analyses as described in FIG. 2. For example, for multiomic analysis where various analytes of a single cell are analysed. The bead may be used to capture one analyte. The droplet which contains further analytes may then be further manipulated (e.g. using any of the modular microfluidic devices and methods below) to analyse a further analyte from the cell. If various capture agents are present on the bead, i.e. molecules which bind various analytes, these can all have the same barcode (or other identifier, by identifier meaning any molecule capable of identifying the analyte as from the same cell) meaning different molecular entities in a single cell are able to be traced back to that cell despite being separated from the original drop.

As an example one could immobilize proteins and RNA on a bead and have DNA in the supernatant, then the first cleavable capture agent could be cleaved using UV and the second capture agent could be cleaved using chemical release (e.g. reduction) after the first bead extraction.

Modular Microfluidic Device

Picoinjection Module

An example of the module is shown in FIGS. 6 and 7. The module comprises a supply or inlet channel into which the microfluidic droplet is injected. The supply channel also comprises a droplet spacer. The function of the droplet spacer is to add spacer oil to evenly space out the droplets prior to picoinjection. The droplet spacer may comprise an auxiliary channel in fluid communication with the supply channel wherein in use the auxiliary channel is attached to a reservoir of spacer oil.

Downstream of the droplet spacer (by downstream and upstream throughout the specification refers to when in use, the flow through the device in normal use), there is a picoinjector.

A picoinjector is a pressurized channel comprising the reagents to be added to the microfluidic droplet and a pair of electrodes. At the intersection of the picoinjector and the supply channel there are electrodes which generates an electric field to induce droplet coalescence via perturbating the surfactant layer at the droplet interfaces. This allows for the injected solution to be compartmentalized inside the droplet. The injected solution then merges with the rest of the droplet when it moves away from the electrode, in the direction of the flow. By adjusting the adding pressure, the volume of reagent added can be precisely controlled.

The module may additionally comprise a dilution channel upstream of the spacer and in fluid communication with the supply channel. This is shown by example in FIG. 7. The dilution channel is configured to add oil to the droplets upon injection into the device and upstream of the spacer. Diluting oil reduces the packing of the emulsion and prevent shearing of droplets and provide smooth arrangement of droplets in the narrowing chamber before they are evenly spaced by spacing oil. The dilution channel may be upstream of the injection port as shown in FIG. 7.

The module may further comprise a sorter, the sorter for sorting droplets into a first droplet set and a second droplet set. The sorter may comprise a bifurcated sorting junction downstream of the picoinjector. Therefore, once the droplets have been picoinjected, the supply channel splits into two different channels: a first exit channel and a second exit channel. Mechanisms and ways to sort cell populations are set out below for the droplet generation module. These apply equally to the sorter incorporated into the picoinjection module. The module may also comprise additional auxiliary channel providing spacing oil (denoted (4) in FIG. 21b) between the pico-injector and the sorter for additional spacing of droplets before sorting.

Modular Microfluidic System Incorporating Picoinjection Module

By system is meant two devices or modules which work in tandem. The first device is a droplet generation module. This provides the microfluidic droplets which are injected into the picoinjection or second device above (or alternatively the droplet fusion module as described below). The modular nature of the system allows optimisation of each part of the method. Therefore, a module is a device adapted to carry out part of a reaction. The modularity may be provided by physically separate devices.

The droplet generation microfluidic module can use flow focusing, step emulsification or cross flowing droplet formation to form the droplets. Examples of droplet generation modules using flow focusing and having different channel geometries are shown in FIGS. 5, 17 and 27. These are discussed below with reference to FIGS. 5, 17 and 27 only to aid explanation.

The droplet generation module may comprise a droplet generation junction in fluid communication with one or more channels, the channel(s) adapted to flow cells, lysis reagent, a partitioning fluid, e.g. oil, and optionally a bead into the droplet generation junction. The droplet generation junction is adapted to encapsulate a cell, lysis reagent and optionally a bead in the partitioning fluid.

For step emulsification, the droplet generation junction may comprise a microchannel or parallel microchannels that enter the deep outer continuous phase reservoir. The dispersed phase spontaneously breaks into droplets at a step change in the height of a microchannel.

For cross flowing droplet formation, the droplet generator may comprise a T or Y junction. Most commonly, the channels are perpendicular in a T-shaped junction with the dispersed phase (cell suspension, lysis reagent, beads) intersecting the continuous phase (partitioning fluid).

In flow focusing, a partitioning channel is included which is adapted to flow partitioning fluid across the flow of cells, lysis reagent and beads. The partitioning channel may be at an angle, for example approximately perpendicular to the flow of cells, lysis reagent and optionally beads. For flow focusing the junction may additionally comprise a constraint, e.g. a narrowing of the channel which exits the junction which aids shearing of a droplet from the cell suspension, lysis reagent and optionally bead dispersed phase.

An example of a droplet generation module which uses flow focusing is provided in FIG. 5: “first microfluidic device”. The module comprises an encapsulation channel into which cells or cell structures (for example nuclei), lysis reagent and optionally a bead are injected.

The cells and lysis reagent and beads may be injected (by injected here is mean flowed rather than pico-injected as described in the second device) into the encapsulation channel via different channels. Or the cells may be injected into the encapsulation channel as shown in FIG. 5. The beads may also be injected into the encapsulation channel via the lysis channel.

FIG. 5 shows these different channels (encapsulation (into which the cells are injected), lysis reagent channel as well as a bead channel). The module comprises a partitioning channel into which fluid which aids droplet formation, for example oil, is injected. Downstream of this partitioning channel there is a droplet generation junction. The droplet generation junction is found at the intersection between the partitioning oil channels and the encapsulation channel. The droplet form as the aqueous fluid is pushed through the wall of partitioning fluid formed as it flows into the encapsulation channel.

After the droplet is generated, the droplet can be further processed using the picoinjection module.

The droplet generation module may further comprise a sorter, downstream from the droplet generation junction, the sorter comprising a bifurcated sorting junction downstream of the droplet generation junction, the bifurcated sorting junction in fluid communication with a first exit channel and a second exit channel wherein the bifurcated sorting junction is adapted to sort the droplets into a first droplet set which exits via the first exit channel and a second droplet set which exits via the second exit channel. The module may also comprise additional auxiliary channel providing spacing oil between the droplet generation module and the sorter for additional spacing of droplets before sorting. The purpose of sorting is explained below. For example, the sorting may divide the droplets into those containing the live cells (first droplet set) and those containing more than 1 cell, dead cells or empty droplets containing no cells (second droplet set).

The first channel may further comprise a droplet channel, in fluid communication with the first exit channel and adapted to add empty droplets to the droplets to be analysed to bulk out the sample.

Towards the end of the first and second exit channels of the sorter, the diameter and/or depth of the channels may increase. This is to prevent merging of the droplets which can occur when moving from the small diameter and/or shallow exit channels to a collection device, for example a wide and deep outlet for a tubing or a tip, i.e. without a gradual increase in the diameter and/or depth of the channels towards the end of the channels where the droplets are collected.

The mechanism of sorting may be by pre-staining cells. Using Calcein-AM as shown in FIGS. 5 and 17 to stain the live cells, the following protocol can be followed: the light from the 488 nm laser was delivered to a detection spot located upstream of the sorting junction (shown for example in FIG. 5) by the excitation light optical fiber. The emission light emerging from the detection optical fiber connected to the detector tube housing a set of emission filters mounted before the detector of photomultiplier tube. When a fluorescence light signal was higher than an arbitrarily set threshold a high voltage pulse was generated (1 kV) by a set of electronic devices including a high voltage amplifier and delivered to the microfluidic sorting junction by ‘salt electrodes’ filled with 5M NaCl solution. As a result, highly fluorescent droplets with live cells were derailed to the collection channel for positive ‘hits’. The duration and delay of pulse can be modified according to the flow rates and the desired throughput of the sorting.

As an alternative to fluorescence, sorting may be activated by for example: image analysis, light scattering, fluorescence anisotropy, absorbance or Raman scattering activated sorting (including SERS—Surface Enhanced Raman Scattering and SRS—Stimulated Raman Scattering).

The droplets are sorted into the first or second exit channel using for example an electric field (most common) or other forces (magnetic, acoustic, pneumatic, heat).

The width and/or depth of the sorting junction may be the same diameter or larger than the diameter of the droplet. For example, the width and depth of the sorting junction may be 150-200 μm. For example, as a guide, for spinDrop (where the bead is incorporated into the microfluidic droplet in the first step of the method), the encapsulation channel may have a depth of approximately 80 μm; the detection spot may be where the channel is approximately 90-100 μm deep; and the sorting junction may have a depth of approximately 180 μm. These dimensions are a guide for when the cell is a human cell. These example diameters are shown in FIG. 27.

For superDrop, where the bead is incorporated in later steps, the droplets from the encapsulation step are smaller (around 30 μl in volume or 38 μm in diameter). The width and depth of the sorting junction therefore may be approximately 70-120 μm, for example 90-110 μm.

For clarity, the spinDrop and superDrop workflows are as follows:
Superdrop uses the method of claims 10 and 11, implemented with the device of claims 1 and 6. Further modifications of these methods and devices are in accordance with the claims and description.
Spindrop uses the method of claims 13, 16 and 19, implemented with the device of claims 2 and 6. Further modifications of these methods and devices are in accordance with the claims and description.

Therefore, the width and/or depth of the sorting junction may be 1-3 times the diameter of the droplet.

By making the sorting junction deeper and/or wider, this allows more efficient droplet sorting as the droplets can be pulled by the electric field for example more efficiently meaning higher throughput.

The deeper sorting junction (the sorting junction with the larger width and depth compared to the droplet diameter) can be applied to any droplet sorter, not only the sorting devices described in connection with the modular devices above. Therefore, also described is a droplet sorter where the sorting junction is larger than the diameter of the droplet comprising the cell. This general droplet sorter may additionally have any of the features described above for the sorter in connection with the droplet generation module.

Method for Single Cell Analyses

There is also provided a modular method for single cell analyses, the method comprising
a) encapsulating a cell or cell structure, lysis reagent and a bead in a microfluidic droplet;
b) incubating the droplets to release the content of the cell or cell structure; and
c) picoinjecting a reagent into the microfluidic droplet.

By modular is meant different steps of the reaction are carried out independently. This allows optimisation of each step without having to balance one reaction with the requirements of a second reaction.

By cell in this method or in any of the other methods of devices is meant an intact cell.

The cell may be from a eukaryotic or prokaryotic organism.

By cell structure is meant a nucleus, or any other organelle which comprises RNA.

As an alternative to a cell, a virion or virus capsule may also be analysed using the methods and systems of the invention. Therefore, virion or virus capsule may be substituted for cell at any point in the claims or the description.

The lysis reagent may comprise lysozyme, when a bacterial cell is being analysed. The purpose of the lysis reagent is to lyse the cell or cell structure to release the contents.

Alternatively, the lysis reagent may comprise a protease, for example proteinase K.

Because the lysis step is separate from the reverse transcription step, the lysis reagents and conditions used can be harsher (as they do not need to take into account any damage done to the reverse transcriptase which is instead added after the protease, for example proteinase K, is denatured). Therefore, by using a protease such as proteinase K and higher temperatures more RNA can be released. The harsher conditions also mean that the lysis step can perform reverse-crosslinking which is particularly useful for clinical samples which have been crosslinked.

The amount to be added of each reagent is given below in two ways:
1) The amount to be added using the concentrated mixes of the reagents (i.e. the reagents in the reservoirs coupled to the channels); and
2) The final concentration in the droplet after addition of the concentrated mix. The final concentration allows the amount to be added to be calculated accurately, which varies according to the size of the droplet which is in turn determined by if a bead is present and the size of the cell.

The following concentrations are for 2), the final concentration of the reagents in the droplet:

The lysis reagent used may result in a protease concentration of 1-20 U/ml in the droplet. For example, 1, 2, 3, 4, 5, 6, 7, 8, 9, 10, 11, 12, 13, 14, 15, 16, 17, 18, 19, 20 U/ml.

The lysis reagent may also comprise a non-ionic detergent, for example IGEPAL CA360. The concentration of the reagent used may result in a concentration of 0.1-0.5% v/v. For example, 0.1, 0.2, 0.3, 0.4 or 0.5% in the droplet.

By “in the droplet” is meant after encapsulation/droplet generation.

The following is 1) the concentrated mix for use in the droplet generation module:

TABLE 1

| Reagent | Concentration of concentrated reagent |
|---|---|
| Lysis reagent | 0.5-60 U/ml of protease |
| | 0.15-1.5% non-ionic detergent |
| | Preferred: |
| | 4-12 U/ml of protease |
| | 0.35-0.7% non-ionic detergent |

The amount of concentrated lysis reagent added can be approximately 10-70% of the final droplet, for example 30-50% of the final droplet, for example 10, 15, 20, 25, 30, 35, 40, 45, 50, 55, 60, 65 or 70% of the final droplet. This can be implemented by varying the flow rates of the lysis reagent, cell suspension and bead (if present) into the droplet generation junction.

The cell may be present as a cell suspension. The cell suspension may comprise a density gradient medium, for example Optiprep™, which prevents cell sedimentation. The concentration of the density gradient medium in the droplet after encapsulation may be 1-15%, for example, 1, 2, 3, 4, 5, 6, 7, 8, 9, 10, 11, 12, 13, 14 or 15%, optionally approximately 3-8% for whole cells and 1-4% for nuclei.

Cell density in the suspension may be measured by manual or automated cell counting. As a guide, 1 cell may be encapsulated every 10 droplets. The cell concentration in the cell suspension depends on the flow rates and the final droplet volume. This can be calculated as follows (as a guide only):

For example if the droplet volume is 1 nl, and the cell mix is 45% of the final volume in the droplet, load 1 cell per 10 nl/0.45==>2.22 cells per 10 nl==>222 cells per ul of cell mix==>222,000 cells/ml of cell suspension.

The lysis reagent may additionally comprise any one or more of the following: a buffering agent for enzyme stability, e.g. Tris-HCl at approximately pH 8 and/or PBS, salt, e.g. KCl, dNTPs, DTT, EDTA and Tween-20.

Incubation of the droplet comprising the lysis reagent may be at least at room temperature (18-25° C.) for 5-60 minutes followed by at least 70° C. for at least 5 minutes, or further up to 80° C. or 85° C. for at least 5 minutes. This incubation releases the cell or cell structure content into the droplet.

The same lysis reagents and concentrations, and conditions apply to steps a) and b) of the other modular method of single cell RNA analyses, the method comprising:
a) encapsulating a cell or cell structure, and lysis reagent in a microfluidic droplet;
b) incubating the droplet to release the RNA;
c) combining the droplet with: i) a bead; and ii) reverse transcriptase reagent using droplet fusion.

The picoinjection step c) of the method for single cell analyses may comprise picoinjecting 0.0001 nl-2 nl, for example 0.001, 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.7, 1.8, 1.9 or 2 nl; or 25, 30, 35, 40, 45, or 50% of the droplet volume with a reagent. This amount varies with the reagent and the size of the droplet.

The droplet volume can be calculated as follows:

The volume of the droplet can be measured using either of the following methods:
1) Measure radius or diameter on imaging system (like a microscope) and then use formula V= 4/3πR³ to convert to volume.
2) Use the flow rates and calculate droplet generation frequency using a ultrafast camera and divide the cumulative flow rates by the frequency to get the volume per droplet. Method 2 is more precise.

By calculating the droplet volume, the appropriate amount of concentrated mix reagent can be added to achieve the required result. The amount or volume ratio for the lysis reagent refers to that in the final droplet as the droplet is formed during the encapsulation step which encompasses this reagent. The amount or volume ratio can be input into the droplet by adjusting the flow rates of the different reagents into the encapsulation channel to form the droplet.

The method may further comprise a sorting step, downstream of droplet generation. The sorting may divide the droplets into those containing the lysate of live cells (first droplet set) and those containing the lysate from more than 1 cell, dead cells or empty droplets containing no cells (second droplet set). Removing the dead cells, empty cells and/or doublets from the analyses increases the signal-to-noise ratio allowing better allocation of sequencing resources and higher confidence in downstream analyses. The sorting step may be downstream of encapsulation or downstream of picoinjection. The sorter may also remove doublets (droplets containing more than 1 cell). This can be done as the signal from two cells is summed up and, in most cases, is higher than the signal from a single cell. Also, if cells are not located within a close proximity inside a droplet, then the duration of the signal is larger and those long signals can be also discarded during sorting. Fluorescence readout can be also combined with image analysis or scatter light analyses to discard cell doublets and multicellular aggregates.

Specific cell types can be isolated by using fluorescent antibodies or with fluorescent reporter cell lines. This can be done by incubating the cells prior to encapsulation (injection into the first device) with an antibody which binds a subset of the cells. With regards to cell reporter lines, Specific cell types that may harbour intracellular fluorescent proteins or sensors encoding for a desired phenotype can be sorted for enrichment from the pool of cells.

Any of the methods above may be implemented using the modular microfluidic system incorporating the picoinjection module described above or any of the systems described herein. To collect the droplets from the droplet generation module, to facilitate incubation and for reinjection, a collection device may be used with any of the systems or devices described in the form of a kit. The collection device comprises a container, the container comprising an immiscible liquid with lower density than water, optionally a hydrocarbon or silicone oil, the container comprising a tip, wherein the tip is connected to the exit of the droplet generation module and wherein the container is connectable to a pump, wherein the pump optionally aspirates the droplets into the container via the tip;
b) the microfluidic droplets are incubated to lyse the cell; and
c) optionally the droplets are reinjected into the picoinjection device by connecting the container to the pump adapted to eject droplets from the tip.

By using one container to collect, incubate and reinject droplets, this reduces merging of the droplets which can be caused by the droplets travelling along lengths of connective tubing often used for collection. FIG. 8 shows an example of a device where a syringe is used with the syringe pump to re-inject the droplets into the second device. The syringe is filled with an oil compatible with the droplets for re-injection of the droplets back into the second device.

The collection device may be transparent to allow UV radiation to penetrate into the droplets in the device. This allows cleavage of UV-cleavable linkers which bind the primers to the bead.

Methods for Single Cell RNA Analyses

The above modular methods for single cell analyses can be used to analyse single cell RNA. The encapsulation, lysis and incubation steps a) and b) of claims 10 and 13 are as described above. The reagent picoinjected at step c) (in claim 13) or added by droplet fusion (in claim 10) is then one or more reverse transcriptase(s). The concentrated reagents added are as follows:

TABLE 2

| Reagent | Concentration of concentrated reagent |
|---|---|
| Reverse transcriptase reagent | 5-50 kU/ml of reverse transcriptase |
| | 0.2 mM-4 mM dNTPs |
| | Preferred: |
| | 10-25 kU/ml of reverse transcriptase |
| | 0.4-2 mM dNTPs |

The above concentrated reagent may be picoinjected as follows:
a) 0.2-1.5 nl, for example, 0.2, 0.3, 0.4, 0.5, 0.6, 0.7, 0.8, 0.9, 1, 1.1, 1.2, 1.3, 1.4 or 1.5 nl; or
b) 20%-200% the volume of the droplet, for example 20, 30, 40, 50, 60, 70, 80, 90, 100, 110, 120, 130, 140, 150, 160, 170, 180, 190 or 200% of the droplet. Volume of the droplet can be calculated as described above.

Alternatively, the reverse transcriptase reagent is added by droplet fusion in accordance with claim 10.

The volume of reverse transcriptase is calculated to result in a final concentration in the droplet of 1-25 kU/ml, for example, 1, 2, 3, 4, 5, 6, 7, 8, 9, 10, 11, 12, 13, 14, 15, 16, 17, 18, 19, 20, 21, 22, 23, 24 or 25 kU/ml. The concentration of the dNTPs in the droplet after piconinjection may be 0.1-2 mM. For example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2 mM.

The reverse transcriptase may comprise template switching activity. The reverse transcriptase may alternatively bare higher RNA processivity or lower sequence-specific bias. Two or more different reverse transcriptases may be used to produce a mix with both these functions.

The reverse transcriptase reagent may additionally comprise any one or more of the following: divalent metal ion, e.g. MgCl₂, a reducing agent, e.g. DTT, and buffering and salts for stability of the enzymes.

The bead added during droplet generation optionally comprises the plurality of poly-T primers, each primer optionally having a barcode and/or a UMI. The barcode and UMI may also be added to the 5′ end of the mRNA. This also applies if the bead is added during droplet fusion. Further optional features of the bead (for example size; and/or dissolvable or non-dissolvable) are described above on pages 4-5, the section titled “Bead”.

The cDNA synthesis primers may be released from the bead after incubation to lyse the cell. This may be carried out before proteinase denaturation. Release may by UV incubation for example if the cleavable linker to the bead is photo-cleavable as shown in FIG. 1. Alternatively, the cDNA synthesis primers may be cleaved from the bead later in the method. The bead may also be a dissolvable bead not requiring cleavage.

The poly-T primer binds to the polyadenylated tail of mRNAs. Reverse transcription from the 3′ end of the poly-T primer can then be performed.

After picoinjection of reverse transcriptase reagent (or where the reverse transcriptase reagent is added via droplet fusion as described in the alternative device and method below), the droplet may be incubated to allow reverse transcription. Incubation may be at approximately 50° C. for 2 hours followed by approximately 70° C. for 20 minutes.

A cDNA sequencing library is the result. The droplets may then be de-emulsified and sequenced. As the barcodes can be found on each primer on a bead or in a droplet, the barcoded cDNA from a single droplet can be traced back to the single-cell from which it came.

If a second strand of DNA is required for library preparation one or more of the reverse transcriptases may have template switching activity and a template switching oligo (TSO) may be added when picoinjecting the reverse transcriptase (or earlier in the method, for example during encapsulation). The TSO has a primer sequence (i.e. an oligonucleotide sequence suitable for PCR) allowing for PCR directly from the library. The TSO may also comprise a label such as biotin for blocking concatemerization of the TSO; and/or be 3′ locked, for example with LNA to allow stronger binding to the dC tail added by the reverse transcriptase.

For example, 5′-biotin—PCR primer—rGrG(+G)-3′ (where rG is riboguanosine and (+)G is the locked guanosine). This is used for PCR amplification of the cDNA (to provide a second handle), therefore the PCR primer section can be any primer sequence suitable for PCR (i.e. no secondary structure, melting temperature compatible with the barcode, no self-priming or primer-dimer formation). A barcode and/or UMI may also be included in the TSO.

In addition, synthetic RNA spike-in molecules can be added to measure sensitivity and specificity of RNA capture for each droplet. Usually synthetic RNA molecules are not used for droplet microfluidics as all the empty droplets would render that information sequenceable, which renders it too expensive. However, removing empty droplets from the sequenceable pool alleviates this cost burden.

The synthetic RNA molecules can be added at a concentration of 10 molecules-1 million molecules in the final droplet, either during encapsulation or pico-injections or droplet fusion.

The above method of single cell RNA analyses can be implemented using the modular microfluidic system incorporating picoinjection module described above.

Alternatively, as explained the above method for single cell RNA analyses can also be carried out using a high-throughput method for RNA analyses where the picoinjection module is replaced by droplet fusion and the bead is added at the end of the method. This method and the modular system comprising a droplet generation module and droplet fusion module are explained in further detail below.

This method comprises:
a) encapsulating a cell or cell structure, and lysis reagent in a microfluidic droplet;
b) incubating the droplet to release the RNA;
c) combining the droplet with: i) a bead; and ii) reverse transcriptase reagent using droplet fusion.

The workflow of a device for implementing this method is shown in FIG. 17, and when combined with sorting after droplet encapsulation is referred to below as “superDrop”. This method instead incorporates the bead in the last modular step before de-emulsification modular step alongside the reverse transcriptase, using droplet fusion. Therefore, the bead and reverse transcriptase reagent are also provided in a microfluidic droplet which is then fused with the microfluidic droplet from step b).

The encapsulation, lysis reagents and incubation conditions are as described above for the method for single cell analyses. The reverse transcriptase reagent is also as described above for the alternative picoinjection single cell RNA analyses method. The only difference is the volume added of these reagents (as the droplet is far smaller due to the absence of the bead in the first encapsulation step) as follows:

The volume of lysis reagent and/or cell mix may be 10, 15, 20, 25, 30, 35, 40, 45, 50, 60, 70, 80, 90, 100, 110, 120, 130, 140 or 150 pl in the droplet. The concentrations of the lysis reagent in the final droplet and for the concentrated mix are as above for the alternative picoinjection method. By cell mix is meant the volume of cell suspension which is also described above as for the alternative picoinjection method. The total volume of the droplet created by the droplet encapsulation step (where the cell or cell structure is combined in a microfluidic droplet with lysis reagent) may be 10-200 pl, for example, 10, 20, 30, 40, 50, 60, 70, 80, 90, 100, 110, 120, 130, 140, 150, 160, 170, 180, 190 or 200 pl or any range within these numbers.

As explained above for the alternative picoinjection workflow, by calculating the droplet volume, the appropriate amount of concentrated reagent can be added to achieve the required result. The amount or volume ratio for the lysis reagent refers to that in the final droplet as the droplet is formed during the encapsulation step which encompasses this reagent. The amount or volume ratio can be input into the droplet by adjusting the flow rates of the different reagents into the encapsulation channel to form the droplet.

After incubation, these droplets containing the cell lysate are then fused with one or more droplets comprising the reverse transcriptase and bead. The reverse transcriptase and bead may be in 1 droplet.

Example amounts are provided below in Table 3 for a 30 pl droplet formed in the first step of encapsulation.

TABLE 3
SuperDrop guide volumes with reference to FIG. 17

| | Volumes |
|---|---|
| SuperDrop lysis mix | 15 pl of the 30 pl droplet |
| SuperDrop cell mix | 15 pl of the 30 pl droplet |
| SuperDrop RT + bead | 1 nl volume droplet |

The superdrop RT and bead droplet can be made by interfacing closely-packed beads flown in a narrow channel that allows for their compression and the concentrated RT reagent in Table 2 is injected from another channel, resulting in the formation of a droplet containing the RT mix and a bead per droplet at the flow-focusing junction (other droplet generation junctions can be used, for example step emulsification or cross flowing droplet formation. The resulting concentration in the droplet after fusing the two droplets together is as described below.

The volume of reverse transcriptase is calculated to result in a final concentration in the droplet after fusion of 1-25 kU/ml, for example, 1, 2, 3, 4, 5, 6, 7, 8, 9, 10, 11, 12, 13, 14, 15, 16, 17, 18, 19, 20, 21, 22, 23, 24 or 25 kU/ml. The concentration of the dNTPs in the droplet after fusion may be 0.1-2 mM. For example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2 mM.

One or two (or more) reverse transcriptases may be present in the reverse transcriptase reagent. Where two are used, the concentration may be 10 kU/ml approximately of each reverse transcriptase. This concentration may vary as above. For example, 10-15 kU/ml of each reverse transcriptase.

The droplet size to be fused with the cell lysate droplet in step c) of claim 10 (and optionally claim 11), may be 1 nl as described above in Table 3. This is the droplet comprising both the bead and the reverse transcriptase reagent. This droplet may be 50 pl to 2 nl in size, the size depending on the bead used. As a guide, the droplet size will approximately be double the volume of the bead size.

The incubation temperatures and times described above may apply to this high-throughput method. Alternatively, where more than one reverse transcriptase is used, the incubation times may be adapted to each transcriptase. For example, when using both Maxima H minus and Superscript III, the following may be used as a guide: 42° C. for one hour, 50° C. for 30 minutes, ten cycles of 42° C. then 50° C. (2 minutes at each temperature, to remove secondary structures and increase the yield), then 70° C. for 20 minutes.

The method may further comprise sorting the droplets at step a) or step c) as described above. For example, for superDrop, the protocol is as follows:
a) encapsulating a cell or cell structure, and lysis reagent in a microfluidic droplet;
followed by a sorting step downstream of encapsulation step a), wherein the sorting step comprises dividing the droplets into a first droplet set and a second droplet set,
optionally wherein in the first droplet set, the droplets comprise live cells; and wherein in the second droplet set, the droplets comprise dead cells, and/or empty droplets, and/or droplets containing more than one cell or cell structure. The live cells, optionally comprising only one cell or cell structure may then be collected and incubated as in step b);
b) incubating the droplet to release the RNA;
c) combining the droplet with: i) a bead; and ii) reverse transcriptase reagent using droplet fusion.

Downstream reactions such as de-emulsification and optional second strand syntheses and spike-ins are described above.

The modular system for implementing superDrop is described below.

Droplet Fusion Modular System

The high throughput superdrop method above (claim 10 and optionally combined with claim 11) can be implemented using the following system:

A modular microfluidics system for single cell RNA analyses, wherein the system comprises:
a) a droplet generation microfluidic module, wherein the droplet generation microfluidics module is adapted for encapsulation of cells or cell structures and lysis reagent in microfluidic droplets, the droplet generation microfluidic module comprising a droplet generation junction in fluid communication with one or more input channels, the one or more input channels for flowing cells, lysis reagent and partitioning fluid into the droplet generation junction; and
b) a droplet fusion module, the droplet fusion module comprising a fusion junction adapted to fuse the reverse transcriptase reagent and a bead with the microfluidic droplet wherein the droplet fusion module comprises a droplet spacer upstream of the fusion junction.

The droplet generation module is as described above (without the requirement for encapsulation of a bead as this is done with the droplet fusion module in this high throughput method. Therefore, the droplet generation module of the high-throughput device encapsulates the cell suspension mix and lysis reagent but not the bead). The above volumes in Table 3 are for a 30 pl droplet formed by the first droplet generation microfluidic module. The droplet may be 10-200 pl, for example, 10, 20, 30, 40, 50, 60, 70, 80, 90, 100, 110, 120, 130, 140, 150, 160, 170, 180, 190 or 200 pl or any range within these numbers.

These droplets containing the cell lysate (following incubation) are then fused with the droplet comprising the reverse transcriptase and bead in the second module: the droplet fusion module.

Where a sorter is used, as for the superDrop protocol, downstream of encapsulation, live cells comprising only 1 cell or cell structure may be obtained and further processed (by incubating to release the RNA and perform reverse transcription. The droplets comprising dead cells or cells with more than 1 cell or cell structure may be discarded.

Droplet Fusion Module

Droplet fusion uses electro-coalescence at a fusion junction to merge droplets by applying an electric field to destabilize the droplet-droplet interface. By fusion junction is meant the area in the module where the droplets meet and are coalesced.

By adding beads at the last modular step, the method generates smaller droplets after encapsulation. This increases the throughput and the method can proceed faster.

An example of a high-throughput device is shown in FIG. 17. The droplet generation module shown comprises a cell sorter as described above. Following cell lysis to release the RNA (and optional sorting) the droplet is combined with a bead and reverse transcriptase. This is carried out by the droplet fusion module.

The droplet fusion module may comprise a supply channel. The supply channel leads into the fusion junction. Microfluidic droplets comprising: a) the single cell lysate; and b) the reverse transcriptase and a bead, flow into the supply channel as shown in FIG. 17.

Therefore, the module may further comprise: a channel in fluid communication with the supply channel into which droplets comprising single cell lysates may be introduced. These droplets then flow into the supply channel.

The droplet fusion module may comprise one or more further channels in fluid communication with the supply channel which flow microfluidic droplets comprising reverse transcriptase and a bead into the supply channel and from there into the fusion junction. This is shown in the bottom half of FIG. 17.

The module may also comprise a droplet generation junction to form the droplets comprising the reverse transcriptase and bead. The left-hand side of this bottom figure shows a droplet generation junction in fluid connection with the supply channel. The droplet generation junction adapted to form microfluidic droplets comprising the bead and the reverse transcriptase mix. These droplets may then flow into the supply channel. The droplet generation junction may be in fluid communication with one or more channels which supply reverse transcriptase mix, beads and partitioning fluid into the junction to encapsulate the reverse transcriptase and bead in a microfluidic droplet. The droplet generation junction can use flow focusing, step emulsification or cross flowing droplet formation to form the droplets comprising reverse transcriptase and a bead. Alternatively, two separate droplets can be made, one having the reverse transcriptase reagent and the second having the bead.

The module comprises a fusion junction which is adapted to fuse the droplet comprising the cell lysate (depicted as the small droplets) and the droplet comprising the reverse transcriptase and bead (the larger droplets in the fusion junction in FIG. 17) together to form 1 droplet. In the fusion junction the cell lysate droplet fuses with the RT and bead droplet. This can occur due to electro-coalescence or flow-trapping or various other known mechanisms.

The droplet fusion module comprises a droplet spacer upstream of the fusion junction. The function of the spacer is to add spacer oil to evenly space the droplets prior to entry into the fusion junction. The droplet spacer may be downstream of the droplet generation junction. Alternatively, the oil used for generation of droplets with beads and RT might serve as a droplet spacer oil for fusion. Alternatively or additionally, the droplet spacer may add spacer oil between the cell lysate droplets. Therefore, the droplet spacer is in fluid communication with the channel which flows cell lysate droplets into the supply channel. Alternatively or additionally, the droplet spacer may comprise an auxiliary channel in fluid communication with the supply channel wherein in use the auxiliary channel is attached to a reservoir of spacer oil.

The high-throughput device may also have a cell sorter as described above, downstream from the droplet generation junction in the droplet generation module (to sort for example droplets comprising 1 live cell or cell structure from droplets comprising dead cells and/or empty droplets) as well as any of the other features described above for the droplet generation module.

The module may comprise a bifurcated sorting junction downstream of the fusion junction. Therefore, once the droplets have been fused, the supply channel splits into two different channels: a first exit channel and a second exit channel. Mechanisms and ways to sort cell populations are set out above for the droplet generation module.

The droplet comprising the reverse transcriptase and bead may be approximately 1 nl in volume. For example, the droplet may be 0.1-5 nL, for example, 0.2-2 nL in volume. The size will be dependent on the bead used. The fusion of the cell lysate droplet with the second droplet comprising the reverse transcriptase and bead results in a droplet which has a reverse transcriptase concentration (and optionally other reagent concentrations) as described above and in the section “Methods for single cell RNA analyses”.

The supply channel may comprise a channel with a width and or depth larger than the cell lysate droplet but smaller than the RT/bead droplet. This helps the droplets group in pairs for fusion.

Use

Single cell analyses that can be carried out include: analysis of transcriptome, genome, epigenome, proteome, epitome, secretome or metabolome of single cells either by themselves or in combinations (multi-omics).

For example, conventional scRNA-seq, scRNA-seq of specific cell types (based on staining), sequencing of transcriptomes and epitopes (based on CITE-Seq (Cellular Indexing of Transcriptomes and Epitopes by Sequencing) but with fluorescent antibodies instead of barcoded antibodies), total RNA, analysis of small RNA (miRNA and snRNA, snoRNA) from single-cells, multiomic assays (DNA-seq plus RNA-seq or ATAC-seq plus RNA-seq), RNA-seq of interacting cells, SINC-seq, targeted single-cell sequencing.

By “single-cell RNA analyses” is meant the extraction of RNA from a cell or cell structure, for sequencing.

Workflows Using Modular Methods and Devices

The following methods are modular methods which optionally use the modular systems provided above.

### Separating Nuclear RNA and Cytoplasmic RNA for Sequencing

There is provided:

A method of separating nuclear RNA and cytoplasmic RNA for sequencing, the method comprising:
step a) encapsulating a cell and outer membrane lysis reagent in a microfluidic droplet, wherein the outer membrane lysis reagent lyses the outer membrane of the cell;
step b) adding nuclear membrane lysis reagent, wherein the nuclear membrane lysis reagent lyses the nuclear membrane; and splitting the droplet into a plurality of droplets to obtain: i) a droplet with nuclear lysate; and ii) a plurality of droplets with cytoplasmic lysate;
step c) adding reverse transcriptase reagent and sorting the droplets into a first droplet comprising nuclear lysate; and a second droplet set comprising cytoplasmic lysate; and
step d) performing reverse transcription of: i) the nuclear RNA and; ii) the cytoplasmic RNA, wherein the cDNA in the cDNA sequencing library comprises a barcode and optionally a UMI.

This is described in Example 11 and FIG. 20.

By outer membrane lysis reagent is meant a reagent that breaks the outer membrane of a cell without disruption integrity of their nuclei. For example, IGEPAL.

By nuclear membrane lysis reagent is meant a reagent that breaks the nuclear membrane and digests DNA/RNA-interacting proteins. For example, proteinase K.

A bead may be added which comprises primers adapted to initiate reverse transcription. For example, poly-T primers comprising a barcode and optionally a UMI. The barcode is preferably the same for all the primers to allow the cDNA from the nucleus and the cytoplasm to be traced back to the same cell. The bead may be removed prior to splitting the droplet by the bead extraction device described above. Alternatively, the bead may be a dissolvable bead.

Examining Interacting Cells

There is also provided:
A method of sequencing RNA from a plurality of interacting cells, the method comprising:
   step a) encapsulating a first cell and a second cell with a barcoded bead in a microfluidic droplet;
   step b) optionally adding a cell separation reagent;
   step c) adding lysis reagent; removal of the bead from the droplet and splitting the droplet into a plurality of droplets to obtain droplets with lysate from the first cell; and droplets with lysate from the second cell, wherein the RNA in both droplets has the same barcode.

By interacting is meant cells which are physically interacting or non-physically interacting cells which are communicating between each other via signalling.

By cell separation reagent is meant a reagent, for example an enzyme, which separates physically interacting cells. For example, a protease such as trypsin which cleaves interacting synapses between the cells. Alternatively or additionally, Accutase or liberase may be used. Alternatively, the outer membrane lysis reagent described above.

The lysis reagent may comprise proteinase K for whole cell lysis. Alternatively, the cell may be lysed using a two-step protocol to separate the cytoplasmic and nuclear RNA as above.

Very quickly following encapsulation with the lysis reagent, the droplet is split to provide a droplet comprising RNA from the first cell and a droplet comprising RNA from the second cell.

After splitting, the different droplets can be processed in a variety of ways to allow separate sequencing of the RNA from the different cells.

Before splitting, the bead may be removed as described above for the method of separating cytoplasmic and nuclear RNA.

For example, the droplets can be collected and fractionated. That is, the droplet suspension is divided up into individual fractions, for example in different wells in a 96 well plate. The division is done to separate the droplet containing lysate from the first cell from the lysate from the second cell. Reverse transcription can then be performed after de-emulsification in these separate wells.

Alternatively, the droplets may be further processed by the addition of reverse transcriptase and then sorted using for example a bifurcating sorting junction which can identify the different cells. For example, the different cells can be pre-stained as described above.

Alternatively, a unique barcode may be added to each individual fraction or droplet. This allows unique tagging of each droplet. The first barcode added during encapsulation identifies the cells as being the interacting cells. The second unique barcode and third unique barcode allow identification of the different droplets and therefore cells from each other.

These unique second and third barcodes when added to the droplets, can be added when adding reverse transcriptase.

This is described in Example 11 and FIGS. 22 and 23.

Throughout the specification, unless the context demands otherwise, the terms ‘comprise’ or ‘include’, or variations such as ‘comprises’ or ‘comprising’, ‘includes’ or ‘including’ will be understood to imply the method or kit includes a stated integer or group of integers, but not the exclusion of any other integer or group of integers.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country.

### DESCRIPTION OF THE FIGURES

FIGS. 1-4 show the bead extractor and droplet splitter:

FIGS. 1a-c show A microfluidic bead extractor which enables multimodal analysis of single cells.

FIG. 2 shows various modular workflows which can be implemented with the bead extractor device.

FIG. 3 shows the sorted bead and supernatant droplets after splitting with corresponding bead-bound and photocleaved fluorescent probes.

FIG. 4 shows nuclei and cytoplasmic staining using the bead extractor device before and after droplet splitting.

FIGS. 5-16 show the modular workflow comprising better lysis of cells, sorting and subsequent picoinjection of further reagents to perform single cell analyses. In FIGS. 5-16, this is shown with single cell RNA sequencing.

FIG. 5 shows Sorting/Pico-injection—droplet microfluidics workflow overview. Throughput for this operation ranges from 70-120 Hz.

FIG. 6 shows Microfluidic sorting and pico-injection of droplets containing single cells

FIG. 7 shows the picoinjector module used in FIG. 6

FIG. 8 shows the droplet collection device

FIG. 9 (left) shows the sorting outputs after Calcein-AM staining of cultured HEK293T cells.

FIG. 9 (top, right) Green fluorescence signals upon treatment of a Rex-1GFP cell line with and without Calcein-AM.

FIG. 9 (bottom, right) Sorting statistics in the positive and negative channel.

FIG. 10 shows counting results for Microfluidic sorting of droplets containing 1:1 dead/alive HEK293T cells, loaded at λ=0.1 (1×) and λ=0.5 (5×) rates

FIG. 11 shows the sorting results of staining B-cells from mouse splenocytes using PE (IgM, CD19 and CD45R) antibodies

FIG. 12 shows Microfluidic sorting of empty droplets reduces overall noise of background “soup”, yielding a higher proportion of usable reads for downstream analysis

FIG. 13 shows Species-mixing experiment—mESc/HEK293 for both nuclei and whole-cells.

FIG. 14 shows Screening for reaction conditions yielding the highest sensitivity, major representative RNA biotype distribution and coverage of unspliced molecules for increased confidence in computing RNA velocity vectors

FIG. 15 shows an example of the number of UMIs detected for each ERCC molecule and cell

FIG. 16 shows the gene detection rate of invention vs. inDrop and 10× v3 for HEK293T cells

FIGS. 17-18 show a variation of the workflow shown in FIGS. 5-16 which provides Ultra-high throughput, low cost and high-quality single cell RNA sequencing

FIG. 17 shows a workflow overview for ultra-high throughput scRNA-seq

FIG. 18 shows results from the ultra-high throughput scRNA-seq method and device in terms of sorting and species-mixing sequencing assay using mouse ES and human HEK293T cells as an input.

FIG. 19 shows comparisons between current RNA sequencing and the present invention (both normal throughput (as in FIGS. 5-16 above) and high throughput (FIG. 17-18))

FIGS. 20-23 show uses for the modular microfluidic methods and devices provided:

FIG. 20 shows a workflow for separating nuclear and cytoplasmic RNA for sequencing

FIG. 21a shows the picoinjector/bead extractor and droplet splitter used in step 2 of a workflow presented in the FIG. 20. FIG. 21b shows the picosorter used in step 3 of a workflow presented in the FIG. 20.

FIGS. 22 and 23 shows a workflow for sequencing a plurality of interacting cells in a droplet

FIGS. 24-27 show further validation for the efficacy of the described methods and systems, as well as optimisation:

FIG. 24. Number of genes detected for exonic, intronic, exonic and intronic reads using whole-cells, extracted nuclei and fixed whole-cells (human HEK293T cell line) as an input. The order of the type of sample is as follows from the left: Exon: whole-cell; nuclei; fixed whole-cell. Intron: whole-cell; nuclei; fixed whole-cell; and on the right: exon+intron: whole-cell; nuclei and on the very right: fixed whole-cell.

FIG. 25. Mapping statistics for the nascent and non-nascent E8.5 metabolically labelled embryos. (a) Nascent RNA sequenced library, (b) non-nascent part illustrating an enrichment in the proportion of intronic reads in the nascent RNA part.

FIG. 26. Proportion of major cell-types identified in the sorted and unsorted populations of mouse splenocytes, using PE-labelled antibodies (CD45R, CD19 and IgM), underlining an enrichment in the proportion of sequenced B-cells. Labels are ordered in the bar graph starting from the bottom moving to the top of the bar graph: B-cells (for example, these comprise nearly 80% of the sorted cells); T-cells; Macrophages; pDC and the top sections of both bars is NK cells.

FIG. 27: A) Design of the improved spinDrop device with zoomed area (B) depicts a droplet generation, a detection spot and a sorting junction, each with different depth of channels.

### EXAMPLES

Aspects of the present invention will now be illustrated by way of example only and with reference to the following experimentation.

### Example 1: A Microfluidics Device for Droplet Splitting and Bead Extraction

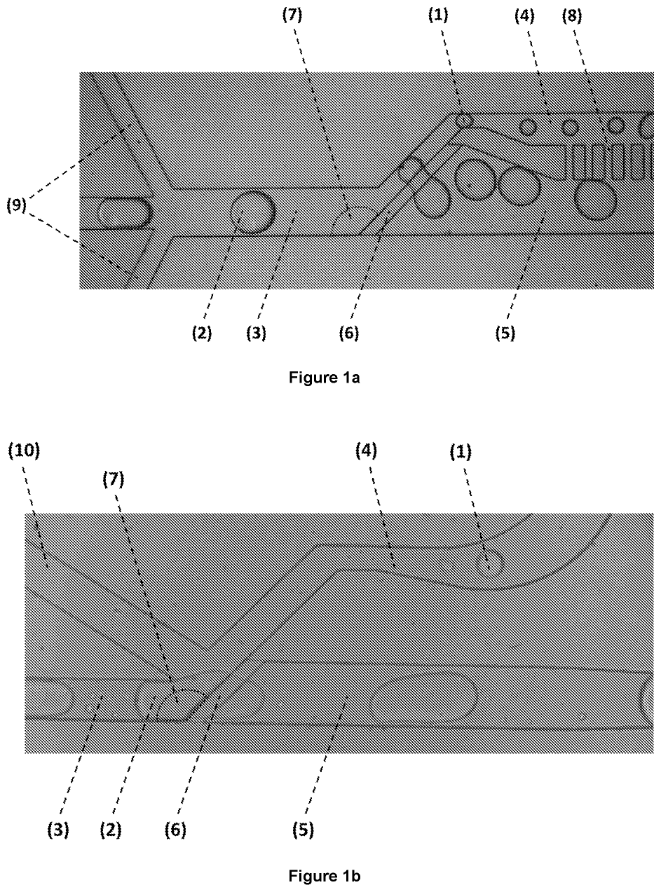

FIG. 1a shows an example of the bead extractor device. A microfluidic device for extracting beads (1) from a microfluidic droplet (2) in a microfluidic junction, comprising a supply channel (3), a first exit channel (4) and a second exit channel (5), and the partial barrier (6) obstructing the access to one of the exit channels. The barrier is at a certain angle (7) to the inlet (supply) channel.

The exit channels can be interconnected by small channels (8) for a pressure equilibration.

There can also be an additional channel or channels (9) connected to the supply channel in order to provide more spacing fluid between droplets and/or to speed up droplets before entering the microfluidic module for extracting the solid objects from the droplets.

The microfluidic droplet is injected into the supply channel by for example syringe pump or other flow, the droplet then travels along the supply channel to the bifurcated junction. The smaller entry into the second exit channel bars the bead from entering this channel. However, the droplet can enter and entry of the droplet into the second exit channel is facilitated by the lower pressure in this channel. The bead surrounded by a very small amount of the droplet (the majority of the droplet volume having entered the second exit channel) is then extracted via the first exit channel.

FIG. 1b shows another example of the bead extractor device comprising most of the same elements as extractor in the FIG. 1a. Number 10 denotes an auxiliary channel adjoining one of the outlet channels. The flow from the auxiliary channel (10) cause the increase of flow resistance in the first exit channel (4).

FIG. 1c shows a scheme of another set up in a bead extractor for reducing the pressure in the second exit channel. Splitting of the droplet from the bead is induced by aspiration of the droplet supernatant. The junction size is smaller than the physical object (bead). (1) denotes the supply channel, (2) the bifurcated junction (3) the second exit channel where aspiration of the solubilized supernatant reduces the pressure to allow the droplet to travel through the entrance and into second exit channel (3) and (4) the bead droplet outlet (first exit channel).

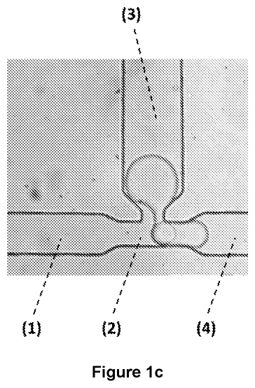

FIG. 2 shows a workflow for multiomic analysis of single cells using bead extractor in droplets. This workflow could be applied to numerous multi-omics protocols. The cell could as an example be lysed in a droplet containing a bead, then the bead is photocleaved in droplets. In an alternative version, a bead can be encapsulated before, photocleaved and then fused with a droplet containing a cell. The RNA is captured on barcoded oligodT on the bead, the bead is extracted and either we inject a RT mix or we de-emulsify and perform RT on a bead. The supernatant contains photocleaved barcoded oligos, and the droplet can be injected multiple times to perform different molecular biology steps (e.g. transposition for open-chromatin profiling and bisulphite conversion for methylome).

Successful extraction of the bead from a droplet using the device was tested as follows: A Polyacrylamide solution consisting of 1 mM Tris-HCl (pH 8), 13.7 mM NaCl, 0.27 mM KCl, 1 mM EDTA, 0.01% Triton X-100, 6.2% Acrylamide, 0.18% Bis-Acrylamide, 50 μM 5′-photocleavable-acrydite oligonucleotide, 50 μM 5′ acrydite oligonucleotide and 0.3% (w/v) Ammonium persulfate was injected in a 60 μM height flow-focusing device and partitioned with surfactant oil (1.5% RAN and 0.4% (v/v) Tetramethylethylenediamine (TEMED) in HFE7500). The droplets were generated to have a diameter of ˜65 μm and solidified overnight in an incubator set to 65° C. The solidified PolyAcrylamide (PAm) microgels were de-emulsified using 40% 1H,1H,2H,2H-Perfluoro-1-octanol and washed with 5 mM Tris-HCl (pH 8), 5 mM EDTA, 0.05% (v/v) Tween-20 (TET buffer). The beads can then be barcoded according to the inDrop procedure and re-suspended and washed three times in hybridization buffer (10 mM Tris-HCl (pH 8), 0.1 mM EDTA, 0.1% (v/v) Tween-20, 332 mM KCl and 10 μM of 5′-Cy5 and 5′-FAM labelled complementary probes were added to the mix containing beads. The mix was incubated at room temperature (23° C.) for 30 minutes, and the excess probes were washed off by three washed with TET buffer. The beads were then encapsulated using a triple-inlet flow-focusing device with 1×PBS, 7.5% (v/v) Optiprep and 0.25% IGEPAL-CA630 and collected in the collection device. 15 pl of droplets were then put aside for imaging (pre-cleavage droplets). The remainder droplets were then illuminated for 6 minutes with a High-Intensity UV Inspection Lamp (UVP). The droplets were then imaged using a Cy5 and GFP emission/excitation filters in a EVOS fluorescence microscope imaging system.

The droplets that were illuminated were then flown in the bead extractor device in order to separate the beads from the droplets. The droplets and beads were then imaged separately using a Cy5 and GFP emission/excitation filters in a EVOS fluorescence microscope imaging system.

### Results Summary

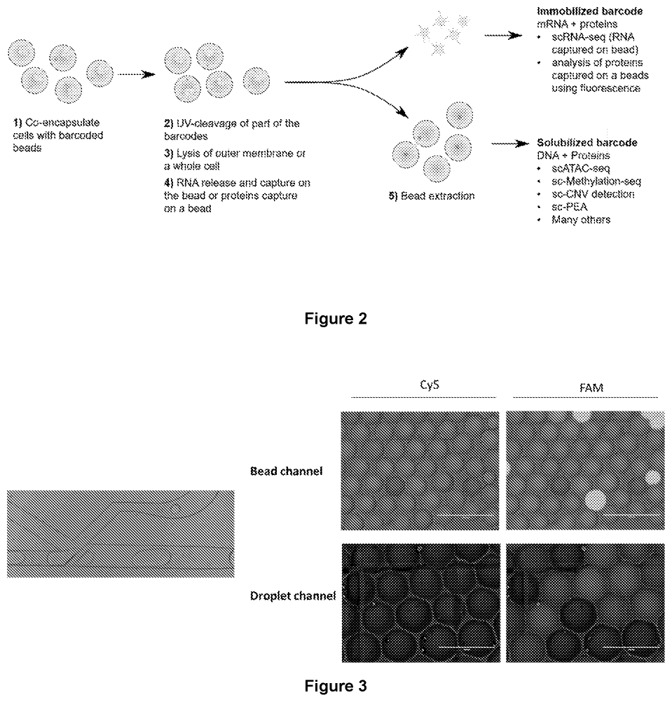

The results are shown in FIG. 3.

FIG. 3 illustrates the droplets extracted from the bead fraction and the supernatant fraction. The photocleavable FAM oligonucleotide can be retrieved from the droplet supernatant, whereas the bead bound Cy5 adapter is found exclusively in the sorted bead droplets, with some residual un-released FAM adapters.

### Example 2: Bead Extractor with Droplet Splitter

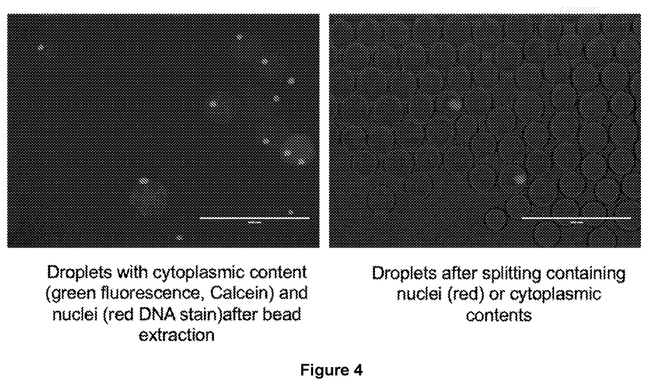

FIG. 4 shows a workflow using the bead extractor and splitter. In the first step a cell is encapsulated with a barcoded bead and photocleave of the barcodes. The cell is lysed and releases cytoplasmic content whereas the nuclei is kept intact. The droplet is then split into 100 small droplets. As the aqueous phase contains a DNA dye, it is then possible to sort out the nuclei fraction and process to RT on both the cytoplasm and nuclei fraction separately (but the barcodes are linked). This workflow would allow elucidation of the mechanisms of RNA localization (in cytoplasm or nuclei).

HEK293T cells were cultured as previously described and stained with 1 μl of far-red fluorescence ethidium homodimer-1 and 1 μl of green fluorescence Calcein-AM per ml of cells for 30 minutes on ice and protected from light. The cells were then co-encapsulated with the lysis mix, without the addition of proteinase K, and barcoded beads as previously described. The droplets were then imaged using an EVOS fluorescent microscope for both green and red fluorescence. The droplets were then re-injected in the bead-extraction module at a rate of 100 μl/hour and the supernatant droplets were split using 2% RAN in HFE-7500 at 2,500 μl/hour and imaged using the red channel on the EVOS fluorescent microscope channel.

Results Summary

FIG. 4 illustrates the ability of separating the cytoplasmic content (green fluorescence yielded by cytoplasmic esterase cleavage) and nuclei (red DNA stain) into separate compartments that contain either released cytoplasmic content, including cytoplasmic RNA, and nuclei droplets, containing mostly nuclei RNA.

### Example 3: Modular Workflow for Single Cell RNA Analyses

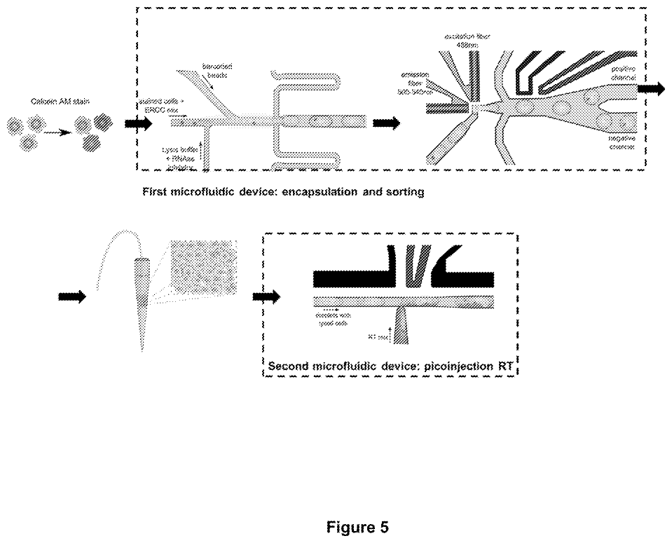

FIG. 5 shows an example modular microfluidic workflow for single cell analyses. The workflow starts by staining the cells (viability stain, antibody or both) or nuclei (DNA stain). Then co-encapsulation of the cells with a barcoded polyacrylamide bead and an improved lysis buffer containing a non-ionic detergent and proteinase K in a microfluidic device (which would degrade any other enzyme in the mix, hence the need for a second step). The droplet is then sorted in a sorting junction using optical read-outs, and the droplets containing a single-cell are collected. The barcode is then photocleaved off and the droplets heated up to 70° C. to unfold RNA secondary structures, to de-activate the proteinase K and to assist with lysis. A reverse transcriptase mix is then picoinjected. This picoinjection step is fast as only the cells that have a cell are injected (due to the sorting step) and this is also cheaper than existing methods, as the RT is only injected in the droplets of interest hence fewer reagents are used. As empty droplets/dead cells are not included in the sequencing there is less background noise in the sequencing results. After reverse transcription, the droplets are de-emulsified and library preparation can proceed.

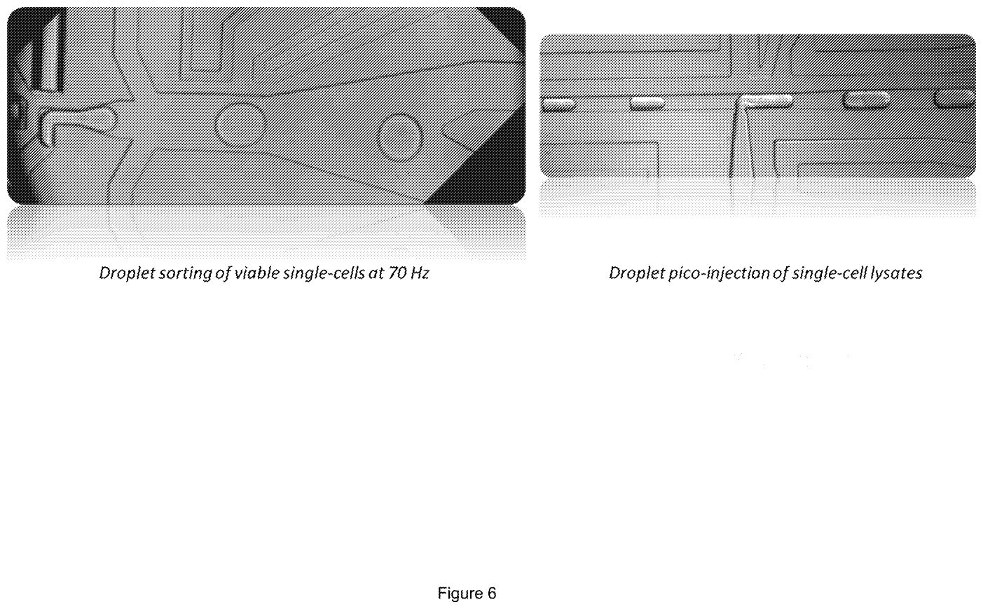

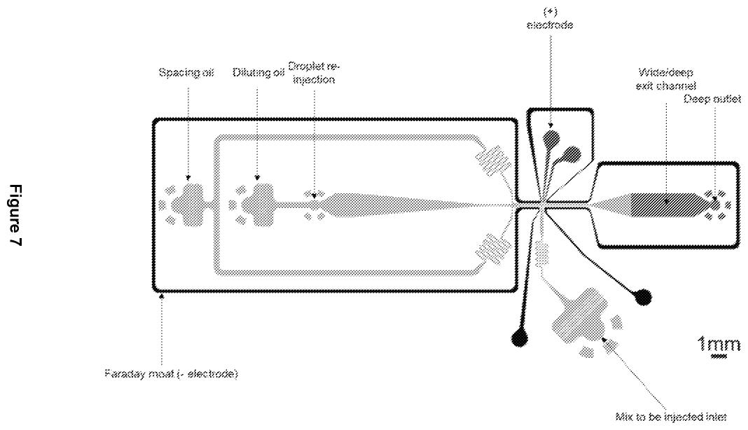

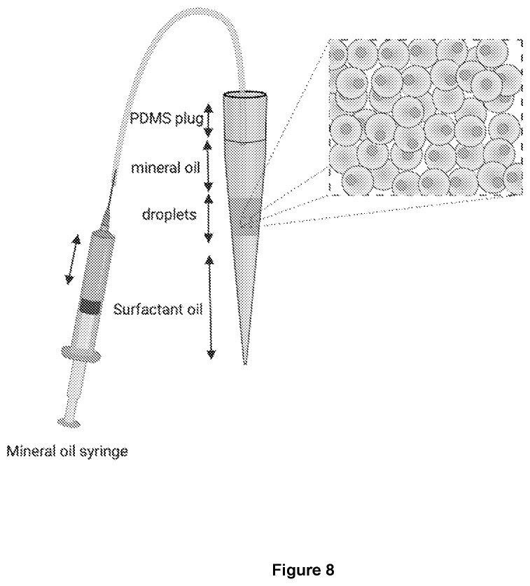

FIG. 6 shows two of the modular aspects of the modular microfluidic system: the cell-containing droplet sorter with bifurcated sorting junction; and picoinjection.

The example below shows the use of this modular microfluidic workflow to sort live cells of interest from empty and dead cells.

HEK293 Ts were passaged every second day and cultured in T75 flasks. The culture media was DMEM (4500 mg/L gluc & L-glut & Na bicarb, w/o Na pyr, D5796-500 ML, Sigma) supplemented with 10% heat-inactivated FBS and 1× Penicillin-Streptomycin. For passaging and collection, the cells were washed with 10 ml of ice-cold 1×PBS (Lonza) twice. 9 ml of PBS was added to the flask and cells were detached by adding 1 ml of 10× Trypsin-EDTA (Sigma-Aldrich) and incubated at 37° C. for 5 minutes. Trypsin-EDTA was then inactivated with 15 ml of DMEM 10% FBS and incubated at 37° C. for 5 minutes. The cells were then pelleted at 300 g for 3 minutes and the supernatant was aspirated. For the experiment, 1 pl of Calcein-AM and 1 pl of ethidium homodimer-1 were added to one ml of washed HEK293T cells and incubated on ice for 25 minutes. The cells were then pelleted at 500 g for 5 minutes at 4° C. and res-suspended in 1×PBS, and brought to a concentration of 250 cells per μl for 1× loading and 1,250 cells per μl for 5× loading. The cells were then mixed 1:1 with a solution of 1×PBS+30% (v/v) Optiprep and encapsulated using the sorting device described and replacing the bead and lysis mixes with PBS. The droplets containing live/dead/empty/doublet droplets were then evaluated using a EVOS fluorescent microscope.

To compare the fluorescent signals obtained from Calcein-AM stained cells and fluorescence from reporter cell-lines, Rex-1 GFP mouse embryonic stem cells were cultured in 2i+LIF (Dulbecco's Modified Eagle Medium F-12 (DMEM/F-12) Nutrient Mixture, without L-Glutamine and Neurobasal Medium without L-Glutamine in a 1:1 ratio, 0.1% Sodium Bicarbonate (7.5%), 0.1% Bovine Albumin Fraction V Solution (7.5%), 0.5× B-27 Supplement (50×), 0.5× N-2 Supplement (100×), 0.1 mM 2-Mercaptoethanol (50 mM), 2.2 nM L-Glutamine (200 nM), 110 U/ml Penicillin-Streptomycin (10,000 U/ml), 20 μg/ml Insulin Zinc (4 mg/ml), 0.2 μg/ml mLIF (10 μg/ml), 3 μM CHIRON99021 (10 mM), 1 μM PD0325901 (10 mM)) and harvested using trypsin. The cells were then counted and re-suspended as previously mentioned and encapsulated in the device shown in FIG. 5. The fluorescent signals were then measured using the emission fiber for both Calcein-AM and non-Calcein-AM treated Rex-1 GFP cells.

Results Summary

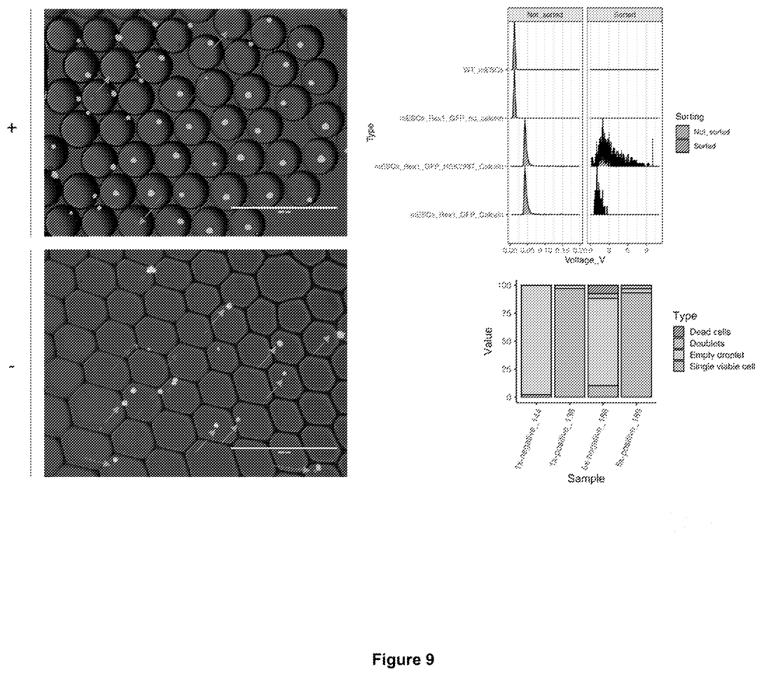

The results are shown in FIG. 9.

Here we demonstrate the modular method's suitability for isolating single-viable cells from the empty/doublet and dead cell droplets using in-line droplet sorting. At 1× concentration concentration (λ=0.1 which means 1 cell per 10 droplets on average), the droplets carrying one viable single cell constituted 98.1% of the droplet pool. Consequently, the ability to perform super-loading of the cells in microfluidic droplets (5× loading, 1 cell every two droplets λ=0.5 which means 1 cell per 2 droplets on average) is demonstrated as 93.1% of the droplets contain single viable cells. Furthermore, the method can distinguish viable-cells from reporter-cell line signals, as the signal given by viability staining supersedes greatly the fluorescence emitted by the Rex-1 GFP mES cell-line.

### Example 4: Ability to Sort Cells in Modular Microfluidic Device Confirmed with Spike-In Experiment

HEK293T cells were cultured and harvested as previously mentioned and the dissociated single-cells were either treated with 1×PBS, 0.25% IGEPAL-CA630 or with 1×PBS for 20 minutes on ice. The IGEPAL-CA630 treated cells resulted in 20% observable viability. The lysed and intact cells were then pooled and stained using Calcein-AM and ethidium homodimer-1 and the concentration of cells was adjusted to a 250 cells per μl in 1×PBS, 15% (v/v) Optiprep for 1× loading (λ=0.1 which means 1 cell per 10 droplets on average) and 1,250 cells per μl for 5× superloading (λ=0.5 which means 1 cell per 2 droplets on average). To enable detection of nuclei with green fluorescence, a part of the pool was also treated with 1 μl of Vybrant DNA stain and superloaded. All three pools were then encapsulated and sorted using the in-line encapsulation and sorting microfluidic device. The droplets were then evaluated and outcomes were counted on a haemocytometer using a EVOS fluorescent microscope.

Results Summary

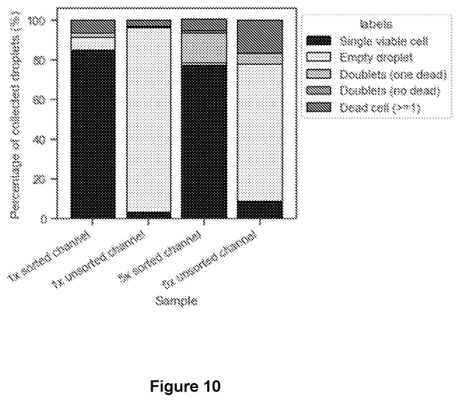

The results are shown in FIG. 10.

FIG. 10 demonstrates the potential for in-line sorting to remove dead cells and empty droplets from the pool of sequenceable cells by performing a 1:1 dead/alive spike-in experiment. The droplet collection and analysis after sorting indicates that for 1× loading, 84.8% of the droplet pool contained single viable cells.

### Example 5: Enrichment of Specific Cell Types for Single-Cell RNA-Seq Using the Modular Workflow

Frozen Splenocyte vials from C57BL/6 mouse were thawed in a water bath at 37° C. and immediately pipetted in 13 ml of pre-warmed (37° C.) 1×PBS, 10% FBS. The mix was spun down at 300 g for 5 minutes at 4° C. and washed twice with 1×PBS 2% FBS. The cells were then strained through a 50 μm cell strainer and 1 million cells in 100 μl were incubated with 10 μl of Fc receptor block (Miltenyi Biotec) for 10 minutes on ice. 2 μl of PE-anti IgM, CD19 and CD45R antibodies (Miltenyi Biotec) were added to the mixture and the cells were further incubated on ice for ten minutes. The cells were then washed two additional times with ice-cold PBS and counted and encapsulated as previously described, without the addition of proteinase K and IGEPAL-CA630 to avoid cell lysis. The laser used for detection was diode pumped solid-state (DPSS) Coherent OBIS 561 nm 50 mW, with an excitation of 561 nm and the emission was captured through a 1× notch filter 561 nm plus 2× bandpass 593/40 filter. The droplets from each sorting channel were imaged under an EVOS fluorescence microscope.

Results Summary

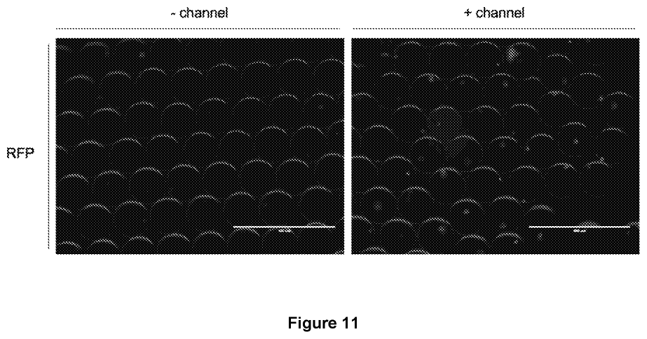

The results are shown in FIG. 11

FIG. 11 shows the enrichment for immunostained B-cells from a mouse splenocyte frozen sample, illustrating the capabilities of the modular microfluidic system to enrich for specific cell types from the input population of cells.

### Example 6: Modular Microfluidic Device Allows Higher Quality Single-Cell Libraries

mESc were cultured and prepared for encapsulation as previously described. Half of the cells were processed as per the inDrop protocol (Zilionis, Nature Protocols, 12, 44-73 (2017)), the other half was stained with Calcein-AM (1 μl per ml of cell suspension) and the stained cells were loaded in the cell loading channel of the in-line sorting device at a concentration of 250 cells/μl in 1×PBS, 15% Optiprep. The lysis mix and bead mix were kept identical to the conventional inDrop protocol, but the droplets containing viable cells were extracted and collected in a 1.5 ml LoBind Eppendorf tube pre-filled with 200 μl of mineral oil. The libraries were then prepared as per the inDrop protocol and sequenced on a Nextseq 75 bp High Output Illumina kit (Read 1 61 cycles, Index 1 & 2 8 cycles each and Read 2 14 cycles). The reads were then inspected and QC'd using FastQC. The data was then demultiplexed using Pheniqs and mapped and counted using zUMIs. Read per barcode plots were obtained from the pipeline directly.

For reference, amounts used and concentrations of the various reagents are provided below as a guide only:

TABLE 4
Guide amounts and concentrations for device shown in FIG. 5

| | | | | | | | IGEPAL CA- | | Thermolabile | | |
|---|---|---|---|---|---|---|---|---|---|---|---|
| | Tris Hcl ph 8 | | KCl | | NaCl | MgCl2 | 630 | dNTPs | Proteinase K | DTT | |
| cell mix | 0 | | 0 | | 0 | 0 | 0 | 0 | 0 | 0 | |
| spinDrop | 120 | mM | 0 | | 0 | 0 | 0.6% | 3.15 mM | 7.2 U/ml | 0 | |
| lysis mix | | | | | | | | | | | |
| spinDrop | 55 | mM | 75 | mM | 0 | 0 | 0.1% | 0 | 0 | 0 | |
| bead mix | | | | | | | | | | | |
| spinDrop after | 59.3 | mM | 10.7 | mM | 0 | 0 | 0.27% | 1.35 mM | 3.1 U/ml | 0 | |
| encapsulation | | | | | | | | | | | |
| spinDrop | 90.8 | mM | 136 | mM | 0 | 8.1 mM | 0 | 0.47 mM | 0 | 17 | mM |
| RT mix | | | | | | | | | | | |
| spinDrop | 76.5 | mM | 80 | mM | 0 | 4.4 mM | 0.15% | 0.87 mM | 1.4 U/ml | 9.3 | mM |
| final droplet | | | | | | | | | | | |
| after RT | | | | | | | | | | | |
| pico-injection | | | | | | | | | | | |

| | | | | | Maxima | | | Super- | | | |
|---|---|---|---|---|---|---|---|---|---|---|---|
| | | | | | H-minus | | | script | | | |
| | PBS | Optiprep | RnaseOut | | RT | | BSA | III | GTP | TSO | PEG8000 |
| cell mix | 1x | 15% (7.5% | 0 | | 0 | | 0 | 0 | 0 | 0 | 0 |
| | | for nuclei) | | | | | | | | | |
| spinDrop | 0 | 0 | 0 | | 0 | | 0 | 0 | 0 | 0 | 0 |
| lysis mix | | | | | | | | | | | |
| spinDrop | 0 | 0 | 0 | | 0 | | 0 | 0 | 0 | 0 | 0 |
| bead mix | | | | | | | | | | | |
| spinDrop after | 0.43x | 6.43% | 0 | | 0 | | 0 | 0 | 0 | 0 | 0 |
| encapsulation | | | | | | | | | | | |
| spinDrop | 0 | 0 | 2 | kU/ml | 18 | kU/ml | 0 | 0 | 0 | 0 | 0 |
| RT mix | | | | | | | | | | | |
| spinDrop | 0.2x | 2.9% | 1.1 | kU/ml | 9.8 | kU/ml | 0 | 0 | 0 | 0 | 0 |
| final droplet | | | | | | | | | | | |
| after RT | | | | | | | | | | | |
| pico-injection | | | | | | | | | | | |

Results Summary

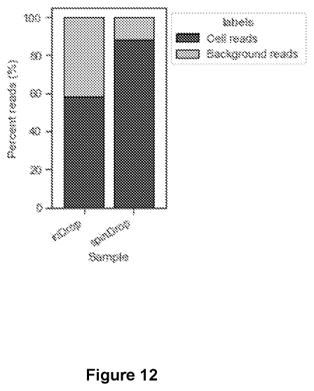

The results are shown in FIG. 12.

FIG. 12 illustrates the potential of in-line sorting to diminish the background noise generated by empty droplets, generated both from circulating extracellular RNA molecules but also primer-dimers or hybrids. This noise materializes in the stacked barplot with increased proportions of unused barcodes (BC) and unmapped reads, respectively. The amount of reads that are useable for contribution to the count matrix are higher with in-line sorting (spinDrop) of viable cells than without (inDrop).

### Example 7: Using Sorting and Picoinjection to Produce High-Quality Single-Cell Libraries From Both Whole-Cells and Nuclei Libraries

mESCs and HEK293T were harvested, and half of the pool was stained with Calcein-AM, according to the manufacturer's instructions. The other half was reduced to a nuclei suspension following the Nuclei EZ prep guidelines and stained for 20 minutes on ice by supplementing 1 μl of Vybrant DyeCycle Green DNA stain to the nuclei re-suspended in 1×PBS, 7.5% Optiprep and 0.04% BSA. The cell solution was loaded at 125 HEK293T cells/μl and 125 mES cells/μl or 125 HEK293T nuclei/μl and 125 mES nuclei/ul. The lysis mix was as follows: 120 mM Tris-HCl (pH 8), 3.15 mM dNTPs (each), 0.6% (v/v) IGEPAL-CA630, 7.2 μJ/ml Thermolabile proteinase K. The inDrop barcoded beads were prepared according to the inDrop protocol (Zilionis, Nature Protocols, 12, 44-73 (2017)), with the inDrop v3 oligonucleotide barcoding scheme. The beads were washed three times in 10 mM Tris-HCl (pH 8), 0.1 mM EDTA and 0.1% Tween-20 and resuspended in 55 mM Tris-HCl (pH 8), 0.1% (v/v) IGEPAL-CA630, 75 mM KCl, 0.05 mM EDTA, 0.05% Tween-20. The solutions were injected in the in-line microfluidic device using 1 ml SGE glass syringes and neMesys 290N pumps and sorted with the following flowrates: 150 μl/hr for the lysis mix, 150 μl/hr for the cell mix, 60 μl/hr for the beads, 700 μl/hr for the main oil, 2,500 μl/hr for the spacing oil. The droplets were collected in a collection chamber pre-filled with mineral oil and incubated at room temperature (23° C.) for 25 minutes. The barcodes were then solubilized from the bead via UV exposure using a high-intensity UV inspection lamp (UVP) for 7 minutes. The collection chamber was then immersed in a water bath solution at 70° C. for 10 minutes and directly immersed in an ice-cold recipient (half part water and half part ice) for 5 minutes after the incubation was finished. The droplets were then re-injected in a pico-injector device and coalesced with a RT solution at 1:1 ratio of flow rates. The RT solution was 1.8× First Strand Buffer (5×FS, Invitrogen), 2.52 mM MgCl2, 9 mM DTT, 3.6 U/μl RnaseOUT, 24 U/μl SuperScript III. The flow rates were the following: 200 μl/hr for the droplet mix, 200 μl/hr for the RT solution, 40 μl/hr for the spacing oil and 400 μl/hr for the main oil. The libraries were then prepared as per the inDrop library preparation protocol and sequenced on a Nextseq 75 bp High Output Illumina kit (Read 1 61 cycles, Index 1 & 2 8 cycles each and Read 2 14 cycles). The reads were then inspected and QC'd using FastQC. The data was then demultiplexed using Pheniqs and mapped to a concatenated mouse and human reference genome and counted using zUMIs. The count matrices were then loaded into Seurat and the number of UMIs and genes for both cell and nuclei fractions were counted and plotted.

Results Summary

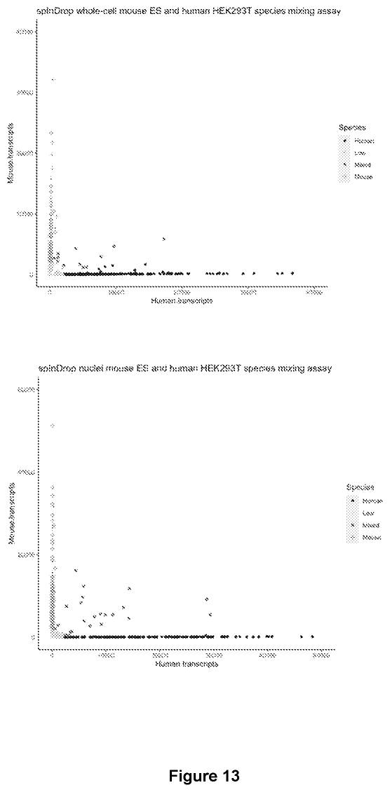

The results are shown in FIG. 13.

FIG. 13 illustrates the potential for generating high-quality single-cell libraries from both whole-cells and nuclei libraries. The low doublet rate in both conditions, 2.9% for whole cells and 5.8% for nuclei demonstrate the ability to generate high-quality, low cross-contamination scRNA-seq libraries using in-line sorting and pico-injection.

### Example 8: Modularisation Allows Optimisation of Each Step of the Method, Giving the Largest Number of Sequence Reads

mEScs and HEK293T cells were prepared and stained with Calcein-AM as previously described and processed with the in-line sorter as described for FIG. 5 with the exception of two lysis mixes were tested, with and without proteinase K added (120 mM Tris-HCl (pH 8), 3.15 mM dNTPs (each), 0.6% (v/v) IGEPAL-CA630, +/−7.2 U/ml Thermolabile proteinase K). Additionally, 259 k molecules of ERCC 92 (Ambion, Mix1) can be added for each ml of lysis mix, to obtain a ERCC read-out. For re-injection in the microfluidic pico-injector, three different mixes were tested: 1) 1.8× First Strand Buffer (5×FS, Invitrogen), 2.52 mM MgCl2, 9 mM DTT, 13.5% PEG8000, 3.6 U/μl RnaseOUT, 24 U/μl SuperScript III that was injected in the droplets not containing proteinase K, 2) 1.8× First Strand Buffer (5×FS, Invitrogen), 2.52 mM MgCl₂, 9 mM DTT, 3.6 U/μl RnaseOUT, 24 U/μl SuperScript III that was injected in either droplets that were treated with and without proteinase K, 3) 1.8×RT buffer (Invitrogen), 2.52 mM MgCl₂, 2 U/μl RnaseOUT, 18 U/μl Maxima H-minus reverse transcriptase. The droplets containing the Superscript III reverse transcriptase were incubate for 2 hours at 50° C. whereas the droplets containing the Maxima enzyme were incubated at 42° C. for 2 hours. The remainder of the library preparation and sequencing analysis was performed as for the previous examples, except that the number of reads per cell was fixed to 20,000 reads per cell for each condition in the zUMIs pipeline. Both intronic and exonic UMI-deduplicated counts were included in the count matrix for comparison.

Results Summary

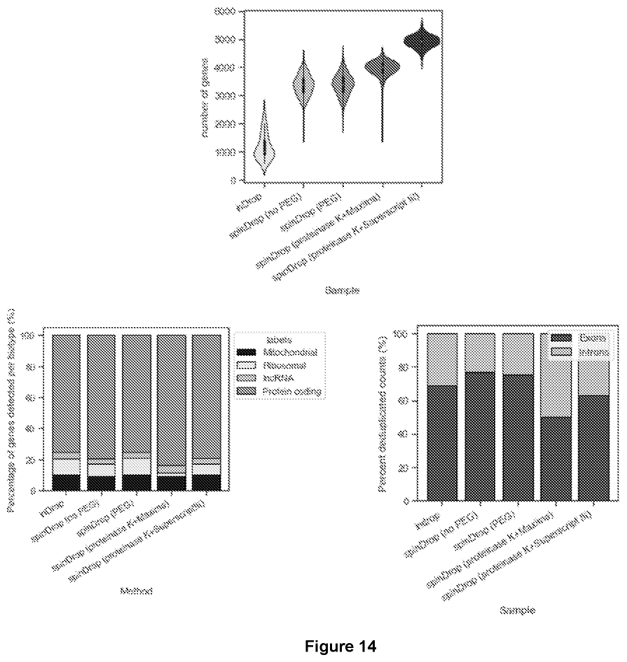

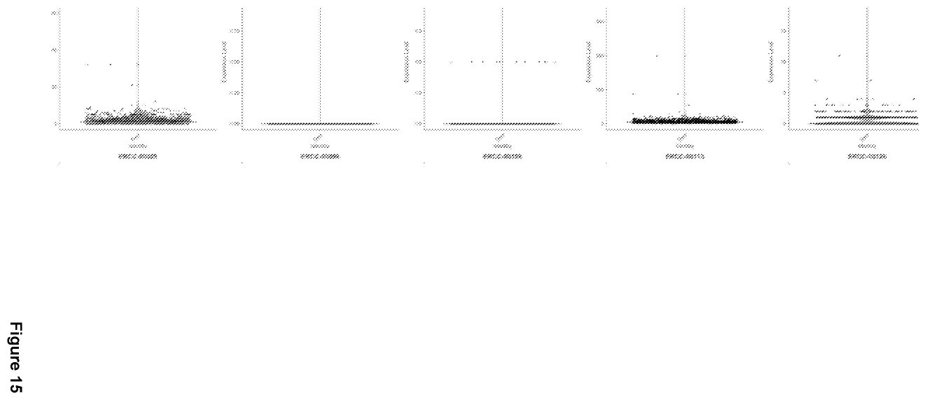

The results are shown in FIGS. 14 and 15.

FIG. 14 illustrates the number of genes that are recovered using a two-step protocol with improved lysis and RT buffer, compared to the inDrop one-step protocol. The median number of genes for HEK293T cells detected using the best performing reaction mixes (Superscript III RT enzyme in conjunction with proteinase K digestion) was 4,926 versus 1,016 for inDrop at a coverage of 20,000 reads per cell, demonstrating the benefit of them method developed to capture larger number of RNA molecules per cell. Surprisingly, molecular crowding with 7.5% (w/v) PEG8000 did not significantly enhance the number of genes detected (3,384 for no molecular crowding vs 4,926 using molecular crowding). Furthermore, the Maxima RT enzyme showed the least enrichment in mitochondrial and ribosomal RNA biotypes compared to lncRNA, demonstrating that this reaction condition likely sampled more molecules from the nuclei. The addition of proteinase K to the mix improved the number of intronic molecules retrieved, likely due to better lysis of the nuclei. Interestingly, the Maxima reverse transcriptase presented the highest fraction of intronic reads 49.8%, confirming a higher capture rate of nuclei-derived RNA molecules, against 31.2% for inDrop. Overall, these results indicate a ˜5× in gene detection efficiency using spinDrop over inDrop, with higher proportions of reads mapping to intronic reads, due to better denaturation of the nuclear envelope. The latter increases the confidence of RNA velocity measurements overall.

FIG. 15 illustrates the capabilities of sequencing ERCC molecules in addition to cellular RNA for each sorted cell using the modular workflow to obtain a capture efficiency metric in terms of specificity and sensitivity using ERCC spike-ins (a subset of the complete ERCC dataset is plotted for visibility).

### Example 9: The Modular Method Gives Improved Results Compared to Current Methods

The fastq files for both spinDrop (with proteinase K and Superscript III) and inDrop conditions datasets generated using HEK29T cells, as well as deposited 10× v3 HEK293T deposited scRNA-seq datasets trimmed to 61 bp for the cDNA read (to match the sequenced lengths of inDrop and spinDrop), were processed with zUMIs at a varying coverages per cell (0; 5,000; 10,000; 15,000; 20,000; 25,000). The count matrices were loaded in Seurat and the median number of detected genes for each method at each coverage was plotted.

Results Summary

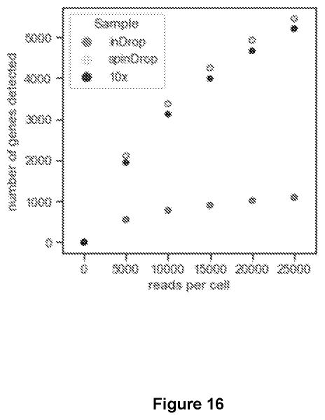

The results are shown in FIG. 16.

FIG. 16 shows the increased capture efficiency of the modular protocol surpasses the current “best-in-class” droplet workflow (10× v3 workflow), and vastly outclasses the capture efficiency of inDrop (five-fold improvement).

In summary, by using a modular workflow, this has allowed optimisation of each part of the experiment:

1) the lysis reagents used do not have to be compatible with the reverse transcriptase. Therefore, more powerful lysis reagents can be used to release all the RNA from the cell. This provides greater capture efficiency and capture of RNA extracted from cell-structures (such as the nuclei).

2) By using a cell sorter, background noise can additionally be eliminated. This background noise results from empty droplets or dead cells. Using the cell sorter also decreases the cost of single cell analyses by only using reagents on droplets containing live cells.

3) Picoinjection allows reliable injection of reagent into the droplets.

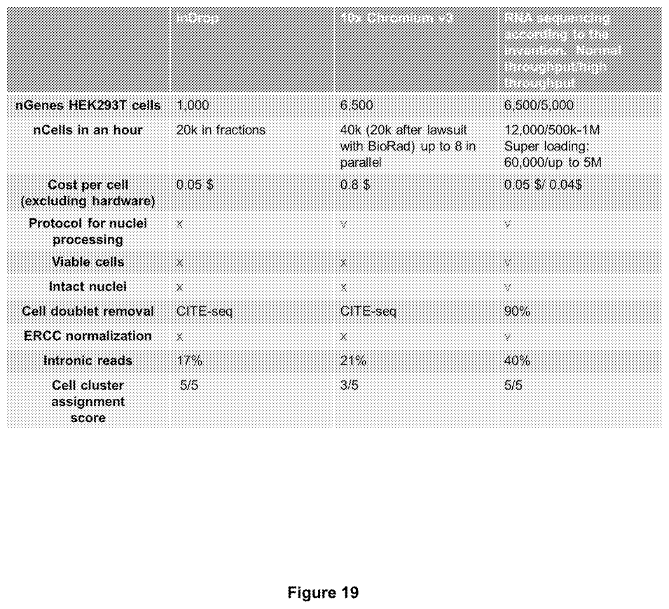

This is shown in improved results over current methods. An overview of the improvements provided by the described methods over various current methods is provided in FIG. 19.

### Example 10: Increased Throughput of the Modular Workflow

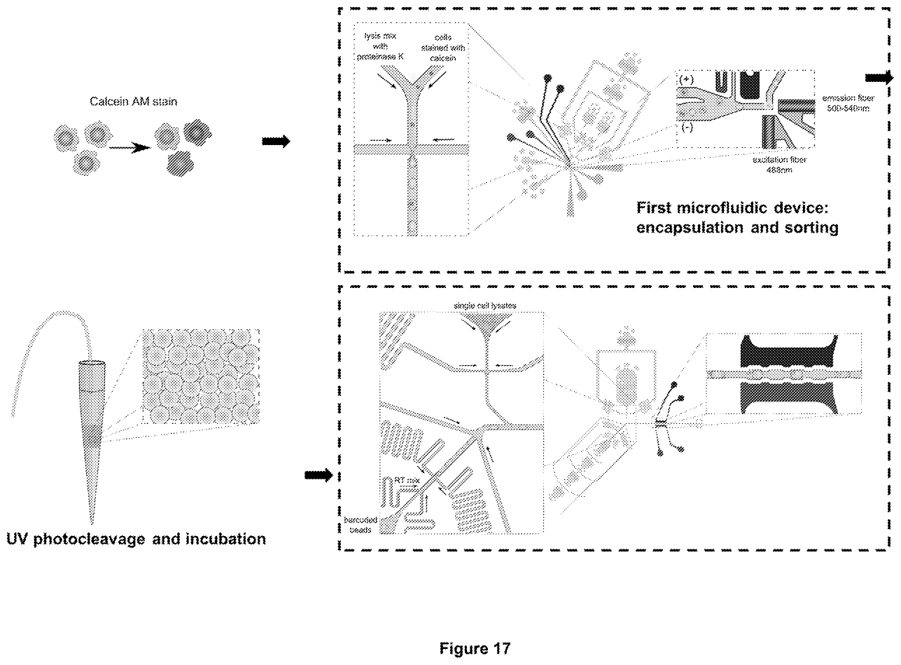

FIG. 17 shows an example alternative modular microfluidic workflow which has even higher throughput compared to that shown in FIG. 5.

The workflow starts by staining the cells or nuclei and injecting them in a microfluidic device with a lysis mix containing a non-ionic detergent and proteinase K. The droplets are then sorted to isolate viable single cells/intact single nuclei at 3.6 kHz. The final merging allows for the fusion of the sorted lysate with a microfluidic droplet containing an optimized RT mix for generating barcoded cDNAs.

An example using this workflow is provided below. Also for reference, and as an example only, the table below sets out in detail the amounts and concentrations of reagents used during the modular picoinjection method used above in Examples 5-8 and the high throughput method of example 10 which uses droplet fusion as a final step.

TABLE 5
Showing the concentrations of the concentrated reagents AND the concentrations of these reagents in the droplet

| | | | | | | IGEPAL CA- | | | Thermolabile | | | | |
|---|---|---|---|---|---|---|---|---|---|---|---|---|---|
| | Tris Hcl ph 8 | | KCl | NaCl | MgCl2 | 630 | dNTPs | | Proteinase K | | DTT | | PBS |
| cell mix | 0 | | 0 | 0 | 0 | 0 | 0 | | 0 | | 0 | | 1x |
| lysis mix | 100 | mM | 0 | 60 mM | 0 | 0.5% | 1 | mM | 12 | U/ml | 2 | mM | 0 |
| mix after | 50 | mM | 0 | 30 mM | 0 | 0.25% | 0.5 | mM | 6 | U/ml | 1 | mM | 0.5x |
| encapsulation | | | | | | | | | | | | | |
| superDrop | 30 | mM | 0 | 36 mM | 5.4 mM | 0 | 0.31 | mM | 0 | | 12 | mM | 0 |
| RT mix | | | | | | | | | | | | | |
| bead mix | 55 | mM | 75 mM | 0 | 0 | 0.1% | 0 | | 0 | | 0 | | 0 |
| RT after | ~30 | mM | 14 mM | 30 mM | 4.5 mM | 0 | 0.25 | mM | 0 | | 10 | mM | 0 |
| merging | | | | | | | | | | | | | |

| | | | | Maxima | | | | | | | |
|---|---|---|---|---|---|---|---|---|---|---|---|
| | | | | H-minus | | Superscript | | | | | |
| | Optiprep | RnaseOut | | RT | BSA | III | GTP | | TSO | | PEG8000 |
| cell mix | 15% | 0 | | 0 | 0.04% | 0 | 0 | | 0 | | 0 |
| lysis mix | 0 | 0 | | 0 | 0 | 0 | 0 | | 0 | | 0 |
| mix after | 7.5% | 0 | | 0 | 0.02% | 0 | 0 | | 0 | | 0 |
| encapsulation | | | | | | | | | | | |
| superDrop | 0 | 1.32 | kU/ml | 12 kU/ml | 0 | 12 kU/ml | 1.2 | mM | 2.4 | uM | 6% |
| RT mix | | | | | | | | | | | |
| bead mix | 0 | 0 | | 0 | 0 | 0 | 0 | | 0 | | 0 |
| RT after | 0 | 1.1 | kU/ml | 10 kU/ml | 0 | 10 kU/ml | 1 | mM | 2 | mM | 5% |
| merging | | | | | | | | | | | |

Polyacrylamide beads were prepared similarly to the inDrop v3 protocol, but with a third cycle of split-and-pool barcoding using similar reaction conditions as in the previous two steps. The lysis mix solution was 100 mM Tris-HCl (pH 8), 6 U/ml thermolabile proteinase K, 1 mM dNTPs, 0.5% (v/v) IGEPAL-CA630, 60 mM NaCl, 2 mM DTT. The cell mix was constituted of Calcein-stained HEK293T and mESc in 1×PBS, 15% (v/v) Optiprep, 0.04% BSA. The loading concentrations were 1,250 cells/μl for each of the cultured cell lines. To prevent sticking of the cells to the walls of the microfluidic device, the cell inlet channel of the microfluidic device were perfused with a coating solution of 1×PBS, 0.5% (w/v) Kolliphor P188 (Sigma), 0.5% (w/v) Pluronic F-127 (Sigma), and 2.5% Tween-20 (Sigma). The flowrate for the cell mix was 100 μl/hr, 100 μl/hr for the lysis mix, 2,600 μl/hr for the main oil, 50 μl/hr for the bias oil and 50 μl/hr for the spacing oil. The cells were collected in a collection tip, incubated at room temperature for 25 minutes and submerged in a water bath at 70° C. for 10 minutes. The container was then submerged in an ice-cold water bath for 5 minutes and the droplets were then re-injected in the droplet merging device. The triple barcoded beads were washed and prepared as previously described and re-suspended in a solution constituted of 55 mM Tris-HCl (pH 8), 0.1% (v/v) IGEPAL-CA630, 75 mM KCl, 0.05 mM EDTA, 0.05% Tween-20. The RT solution was made as follows: 30 mM Tris-HCl (pH 8), 36 mM NaCl, 6% PEG 8000, 1.2 mM GTP, 12 mM DTT, 2.4 μM Template-Switch Oligonucleotide (5′ biotin blocked and 3′ LNA blocked), 0.54 mM MgCl₂, 0.31 mM dNTPs, 1.32 U/μl RnaseOUT, 12 U/μl Maxima H-minus reverse transcriptase and 12 U/μl Superscript III. The flowrates for operating the device were: 35 μl/hr for the bead mix, 250 μl/hr for the RT mix, 300 μl/hr for the generation oil, 200 μl/hr for the spacing oil, 8 μl/hr for the emulsion oil and 1,400 μl/hr for the droplet re-injection oil. The droplets were collected in a 1.5 ml LoBind Eppendorf tube pre-filled with 200 μl of mineral oil. The tube was then subjected to UV exposure for 7 minutes using a High-Intensity UV Inspection Lamp (UVP) and incubated at 42° C. for 30 minutes, followed by 50° C. for 30 minutes and 10 cycles of 2-minute consecutive incubations a 42° C. followed by 50° C. The tube was then finally incubated at 70° C. for 15 minutes and then de-emulsified and stored at −80° C. until final library preparation. Then the aqueous phase was treated with 100 U of exol (NEB) for 30 minutes at 37° C. and AmpureXP purified at 1.2× bead to aqueous solution volume equivalence. The resulting cDNA was eluted in 25 μl and PCR amplified for 13 cycles using a KAPA HiFi mastermix (98° C. for 15 seconds, 60° C. for 20 seconds and 72° C. for 40 seconds). The resulting libraries were SPRI purified with a 1× volume equivalence and ran on a High-Sensitivity Bioanalyzer kit (Agilent) and the DNA concentration was calculated using a Qubit HS kit. 1 ng of the library was subjected to Nextera XT treatment, according to the kit's guidelines and the final PCR was achieved similarly than with the inDrop protocol. The libraries were then purified with 2 rounds of 0.7×SPRI purification and sequenced on a Nextseq 150 bp High Output kit (Read 1 106 cycles, Index 1&2 8 cycles each, Read2 44 cycles). The data was inspected with FastQC and de-multiplexed with Pheniqs. The de-multiplexed data was then mapped on both the mouse and human reference genomes using zUMIs and the resulting count matrices were loaded into Seurat to collect the number of detected genes and UMIs detected got both mESc and HEK293T.

Results Summary

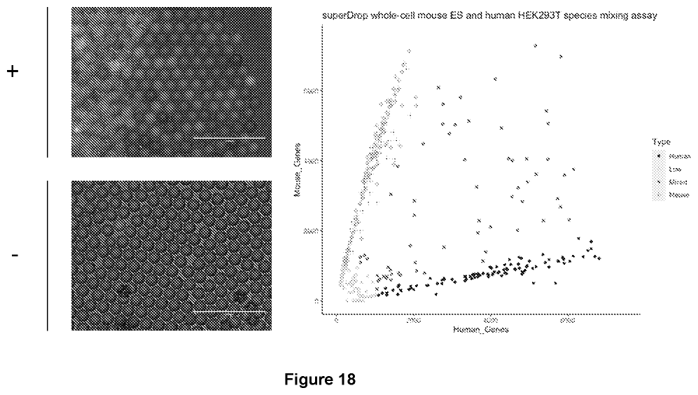

The results are shown in FIG. 18.

FIG. 18 shows the results of sorting Calcein-AM stained mESc and HEK293T species-mixing experiment, after lysis and heat treatment. The positive and negative channel indicate a clear enrichment for viable cells in the positive gate and a depletion of empty droplets. The figure on the right shows the result of species-mixing experiments, in terms of the number of genes that were detected for each barcode by mapping to both species reference genome, showing a clean separation between both cell-types.

By re-arranging the workflow to incorporate the bead into the droplet downstream of lysis and sorting the droplets for live cells, higher throughput can be achieved.

FIG. 19 shows an overall comparison of the methods used in Examples 3-10.

### Example 11: Alternative Workflows that can be Implanted with the Modular Microfluidic Systems

Modular Workflow for Separating Nuclear RNA and Cytoplasmic RNA for Sequencing

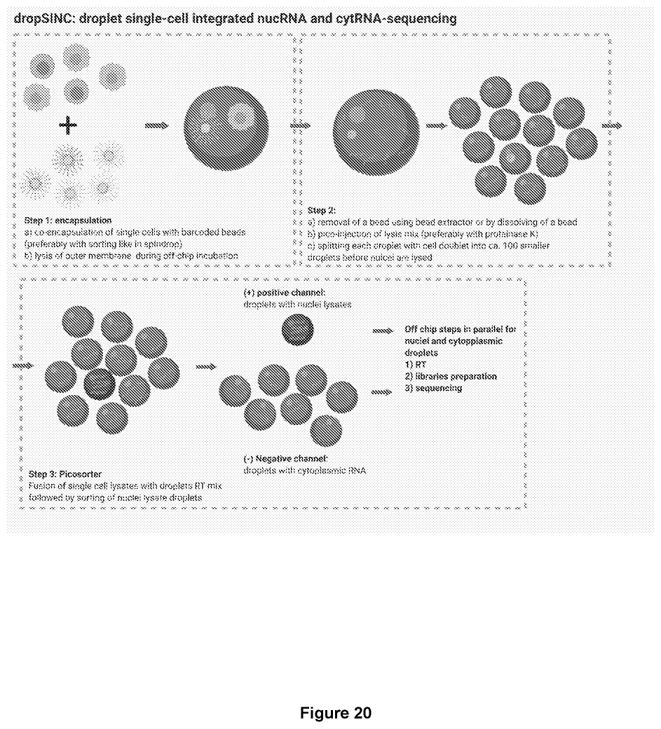

FIG. 20 presents a general dropSINC-seq workflow where we can encapsulate a cell with a barcoded bead and photocleave of the barcodes (alternatively dissolvable bead). The cell is lysed and releases cytoplasmic content whereas the nuclei is kept intact. The droplet is then split into ˜100 smaller droplets. As the aqueous phase contains A DNA dye, we can then sort out the nuclei fraction and process to RT on both the cytoplasm and nuclei fraction separately (but the barcodes are linked). This would allow us to elucidate mechanisms of RNA localization (in cytoplasm or nuclei).

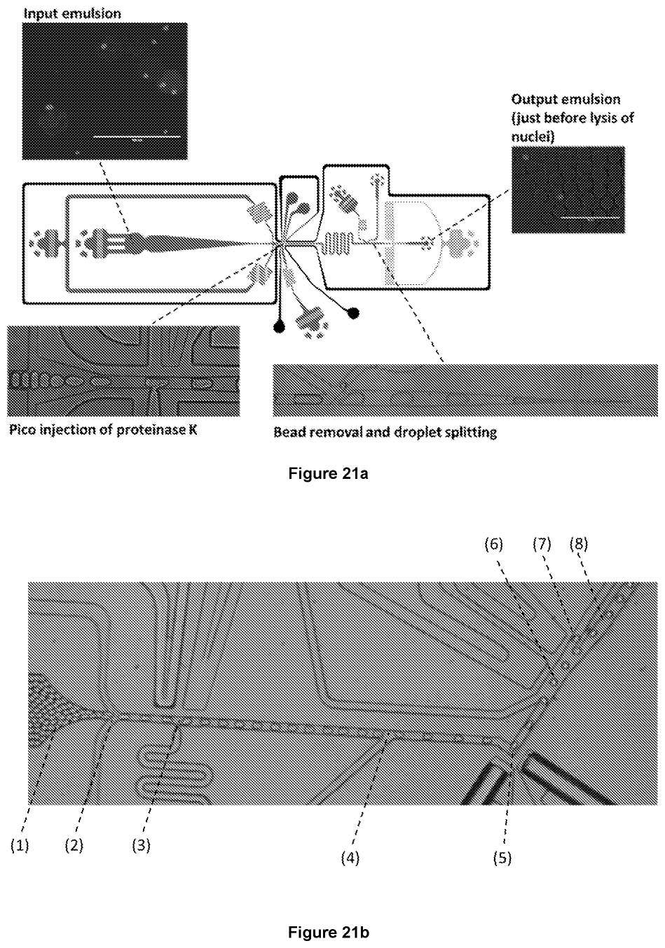

FIG. 21a shows the scheme depicting the operation of microfluidic device for 2nd step of dropSINC. In the first step droplets with single cells with lysed outer membrane (showed as an input emulsion in the left microphotograph, the nuclei are stained with a red dye and cytoplasm with a green dye) and intact nuclei are picoinjected with proteinase K lysis mix. Next, after mixing in winding channel the bead is removed and the droplet is split into c.a. 100 small approx. 20 picolitre daughter droplets containing either nuclei lysate or cytoplasmic RNA. Microphotograph on the right depicts the emulsion after splitting—the nuclei are stained with a red dye and cytoplasm with a green dye.

FIG. 21b shows the third step of a dropSINC-seq protocol. Snapshot depicts the operation of picosorter. Emulsion (1) containing small fraction (less than 1%) of droplets with single nuclei and large fraction (>99%) with cytoplasmic lysate is spaced with oil at re-injection junction (2) and proteinase K mix is pico-injected at pico-injection junction (3). Next, droplets are spaced with additional spacing oil (4) before the fluorescence is measured at measurement spot (5) to trigger the sorting of droplets with single nuclei lysates are at sorting junction (6). Droplets (7) with single nuclei are directed to the positive collection channel, while droplets with cytoplasmic lysates (8) are directed to the negative collection channel.

Modular Workflow for Sequencing the RNA from Two or More Interacting Cells with the same barcode

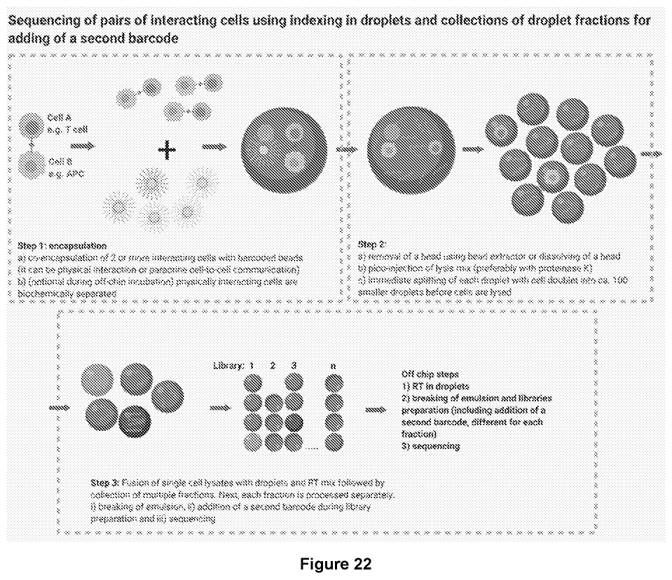

FIG. 22 presents a scheme of general workflow. The droplets can contain either physically interacting cells or single cells (mixture). One could decide to sort the droplets for a dual colour signal to enrich for specific interactions using a dual-colour laser or to process all the droplets in order to obtain physically interacting and non-physically interacting cells together (no pre-sorting needed). In a first case scenario, the cells are further dissociated in the droplet and the barcode is released from the bead. The bead is then removed (via extraction or dissolving) and the droplet is split. Physically interacting cells thus have the same barcode to this point and are now individually compartmentalized in “daughter” droplets. One can then pico-inject either a RT mix or a polyA and RT mix (for 3′ mRNA or total RNA) and proceed to cDNA conversion. The collected droplets are then split into fractions containing multiple droplets before de-emulsification. The number of droplets per cell has to be calculated to keep a possible collision rate (i.e. two physically interacting cells in the same fraction) low. A second barcode can then be added either via ligation, tagmentation, PCR or template switching in the downstream library preparation steps. Another variation for nuclear RNA-seq would be to release the cytoplasmic content of physically interacting cells (that can be enriched for via a dual-colour pre-sorting step or not) in a droplet with a barcoded bead. Release the barcode, extract the bead and split the droplet. The nuclei in each daughter-step will contain a similar barcode if they were interacting. One can then sort the nuclei-containing droplets via DNA staining and follow a similar procedure as before (FIG. 17).

Fundamentally, physical interaction can recover some element of spatial localization in tissues and should allow bioinformatic reconstruction of the spatial organization of the tissue. When spatial architecture is not key (i.e. cells are in suspensions and not tissue), physical interaction is still a key interesting feature, mainly in immunology where one could see which T-cell/B-cell is physically interacting with a cancer cell.

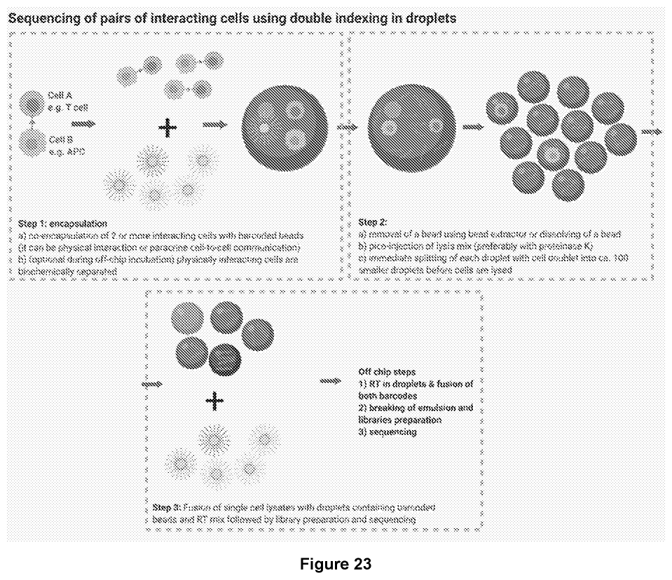

FIG. 23 presents a similar workflow to the assay presented in the FIG. 22. The main difference is the way of adding a second barcode. A way to circumvent fraction collections is to use a merging device during the last RT step to introduce a second barcode (either in aqueous or bead format) that could be either introduced via ligation or template switching. The device would be similar to the second device in the FIG. 17. The first barcode codes for the interacting cells (‘mother droplet’) and the second barcode for a specific cell (“daughter” droplet). If the single cells (i.e. non-interacting) were not discarded initially, they would also receive two unique barcodes.

### Example 12: spinDrop can Perform Reverse-Crosslinking

Because spinDrop can perform reverse-crosslinking in droplets (using a combination of proteinase K and high temperature during the lysis), it can have wide applicability to a range of new sample types, mainly clinical samples or samples that were fixed for pre-treatment (for example metabolic labelling using the single-cell 5EU-seq method).

For this purpose, we generated a set of two results using spinDrop (described in example 7).

First, HEK293T cells were dissociated with TrypLE and washed twice in ice-cold PBS and re-suspended in 200 μl PBS. The cell-suspension was then further fixed by adding 200 μl of 8% paraformaldehyde (PFA) for 10 minutes on a tabletop rotating shaker at room temperature, and further permeabilized by adding 200 μl of 1% Triton X-100 for 5 minutes. The reaction was quenched by adding 500 μl of 1M Tris-HCl (pH 7.5), and the cells were washed twice with PBS supplemented with 0.05% BSA. They were then stained with 1×SYBR stain (Invitrogen), counted and processed using the spinDrop protocol (described in example 7). Exonic and intronic gene counts were provided as an output from the zUMIs pipeline.

Results:

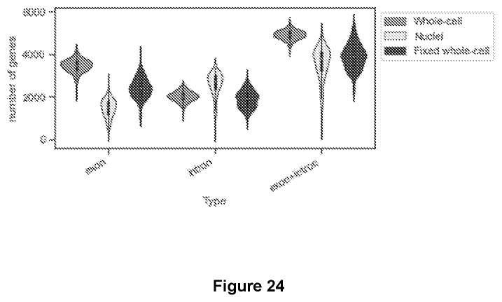

When down-sampling to 20,000 reads per cell, we obtain the distribution of exonic and intronic gene counts for whole-cells (Calcein-AM stain sorting for sorting), nuclei (Vybrant DyeCycle Green DNA stain for sorting) and whole-cells fixed with 4% PFA and permeabilized with 0.25% IGEPAL CA-630 (SYBR stain for sorting), shown in FIG. 24.

The median exonic gene counts were 3,493; 1,533; 2,404 and intronic gene counts were 2,030; 2,714; 1,934 for whole-cells, nuclei and fixed whole-cells respectively. These results indicate high-capture efficiency for a wide range of input material preparation that broaden the accessible range of sample types for scRNA-seq.

### Example 13: Single-Cell 5EU-Seq in Droplets

The ability to differentiate nascent transcripts from mature transcripts enables the prediction of transcriptome dynamics over time. This is helpful to understand complex phenomena that can be observed at the single-cell level, such as differentiation, reprogramming or cell-cycling. One can look at the ratios of un-spliced and spliced molecules using RNA velocity, but the inherent biases in single-cell datasets can blur the complete picture of dynamics in gene expression.

A plethora of methods have been developed to complement RNA velocity measurements using nascent RNA labelling (SLAM-seq or 5EU-seq). Because these labelling methods utilise harsh chemical reactions which are not directly compatible with downstream molecular reaction or use cell fixation methods (which require heat and proteinase K for decrosslinking), they have been largely restricted to plate-based methods.

We have used the single-cell scEU-seq protocol (DOI: 10.1126/science.aax3072) on E8.5 mouse embryos, cultured for 3 hours in IVC1 media (described in 10.1016/j.cell.2014.01.023) supplemented with 500 μM 5EU (5-ethynyl-uridine). The embryos were cut in smaller pieces using sharp blades, and were dissociated with TrypLE for 5 minutes at 37° C. The reaction was quenched using DMEM/F-12 supplemented with 10% FBS and the cells were fixed and pre-processed as per the scEU-seq protocol. The cells were then re-suspended in PBS, counted and processed through the spinDrop workflow (described in example 7) without fluorescence-activated droplet sorting. The downstream library preparation protocol after de-emulsification follows the methodology described in the scEU-seq methodology, however, the final PCR amplification was performed as per the spinDrop protocol, to account for differences in sequencing adapters.

The ability to fix cells and barcode in droplets expands the range of single-cell labelling methods requiring cell fixation that can be ran at high throughput, for example combinatorial indexing methods, or intra-cellular probing of protein contents using barcoded antibodies.

### Results Summary:

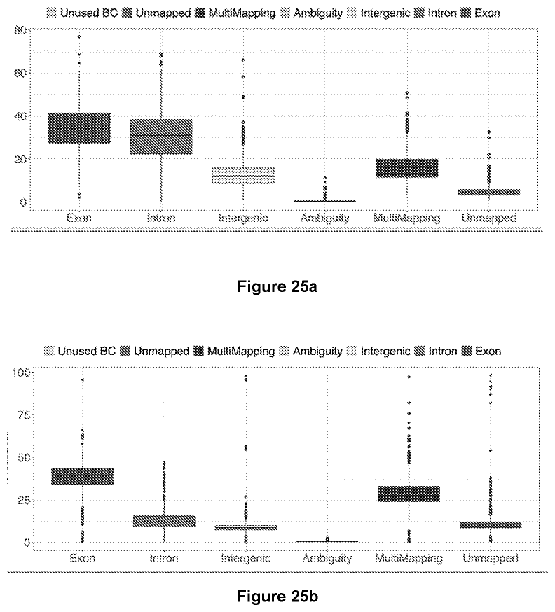

The fraction of intronic reads (marker for RNA being produced) was much higher in the libraries containing the nascent cDNA, obtained via streptavidin pull-down of biotin-labelled EU, which is shown in FIG. 25a, as a result from processing the reads with the zUMIs pipeline. On the other hand, the non-nascent part that was not pulled-down showed significantly higher proportions of exonic reads (FIG. 25b).

### Example 14: Results with Cell Staining Using Antibody Stainings

We also tested the ability of sorting a sub-population of cell-types from a mixed input of cell types, which is useful when numbers of cells are too low for pre-enrichment with FACS followed by high-throughput scRNA-seq.

To achieve this, mouse splenocytes (C57BL/6) were thawed and washed with pre-warmed in 1×PBS with supplemented 10% FBS twice and strained with a 30 μm cell strained. The cells were then washed in 1×PBS with supplemented with 2% FBS and washed once more and 10 million cells were resuspended in 1 ml MACS buffer (PBS, 0.5% FBS, 2 mM EDTA). 100 μl of FcR blocking reagent (Miltenyi Biotec) was added and the mixture was mixed and incubated at 4° C. for 10 minutes. The cells were then further incubated with 10 μl of Cd45R (PE label), CD19 (PE label) and IgM (PE label) antibodies (Miltenyi Biotec) and incubated at 4° C. for 30 minutes. The cells were further washed twice in MACS buffer, and once more in 1×PBS supplemented with 0.05% BSA. The cells were then processed as per the spinDrop protocol (described in example 7), or using the inDrop protocol (as described in https://doi.org/10.1038/nprot.2016.154) as a negative control. The sequencing reads were then processed using the zUMIs pipeline, and marker annotation in Seurat allowed for cluster cell label assignment, and further counting of cells in each cluster.

Results Summary:

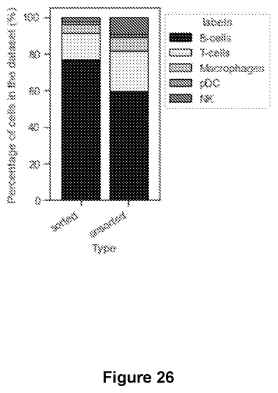

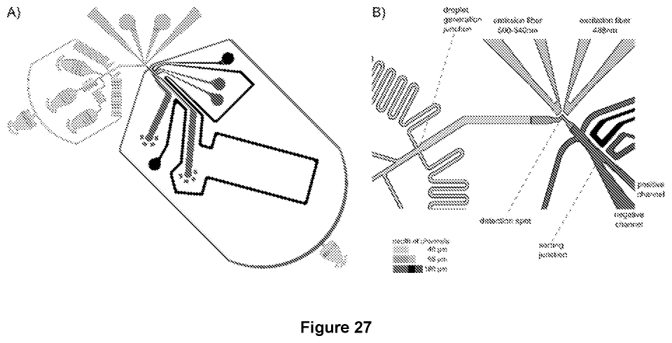

The results are shown in FIG. 26.

The results revealed a 1.2-fold increase in the number of B-cells in the final population of sequenced cells when sorting based on antibody-labelling, compared to unsorted sequencing of the cell population (inDrop). These results underline the capabilities of the workflow to enrich for specific populations of cells using specific fluorescently labelled antibodies, further increasing the desire signal in the sequencing libraries and decreasing the cost.

The project leading to this application has received funding from the European Union's Horizon 2020 research and innovation programme under the Marie Sklodowska-Curie grant agreement No 750772.

The project leading to this application has received funding from the European Research Council (ERC) under the European Union's Horizon 2020 research and innovation pro gramme (grant agreement No 695669).

1. A modular microfluidics system for single cell RNA analyses, wherein the system comprises:
a) a droplet generation microfluidic module, wherein the droplet generation microfluidics module is adapted for encapsulation of cells or cell structures and lysis reagent in microfluidic droplets, the droplet generation microfluidic module comprising a droplet generation junction in fluid communication with one or more input channels, the one or more input channels for flowing cells, lysis reagent and partitioning fluid into the droplet generation junction;
and
b) a droplet fusion module, the droplet fusion module comprising a fusion junction adapted to fuse reverse transcriptase reagent and a bead with the microfluidic droplet, wherein the droplet fusion module comprises a droplet spacer upstream of the fusion junction.

2. A modular microfluidic system, the system comprising:
a) a droplet generation microfluidic module, wherein the droplet generation microfluidics module is adapted for encapsulation of cells or cell structures, lysis reagent and beads in microfluidic droplets, the droplet generation microfluidic module comprising a droplet generation junction in fluid communication with one or more input channels, the one or more input channels for flowing cells, lysis reagent, partitioning fluid and beads into the droplet generation junction; and
b) a picoinjection microfluidic module for single cell analyses comprising:
a) a supply channel, into which microfluidic droplets comprising cell lysate can be injected wherein the supply channel comprises a droplet spacer; and
b) a picoinjector, downstream from the droplet spacer, for injecting reagent into the droplets.

3. The modular microfluidic system of claim 2, wherein the droplet spacer comprises a channel upstream of the picoinjector, and in fluid communication with the supply channel adapted to flow spacer oil into the supply channel.

4. The modular microfluidic system of claims 2-3, additionally comprising a dilution channel upstream of the droplet spacer to dilute the droplets with oil prior to spacing.

5. The modular microfluidic system of claims 2-4, further comprising a bifurcated sorting junction downstream of the picoinjector, the bifurcated sorting junction in fluid communication with a first exit channel and a second exit channel wherein the bifurcated sorting junction is adapted to sort the droplets into a first droplet set which exits via the first exit channel and a second droplet set which exits via the second exit channel.

6. The system of claims 1 and 2, wherein the droplet generation microfluidic module further comprises a bifurcated sorting junction downstream of the droplet generation junction, the bifurcated sorting junction in fluid communication with a first exit channel and a second exit channel wherein the bifurcated sorting junction is adapted to sort the droplets into a first droplet set which exits via the first exit channel and a second droplet set which exits via the second exit channel, optionally wherein the sorting junction has a larger width and/or depth than the diameter of the droplet to be sorted.

7. The system of claim 6, wherein the first exit channel further comprises a droplet channel, in fluid communication with the first exit channel and adapted to add empty droplets to the droplets to be analysed to bulk out the sample.

8. The system of any of claims 1-7 additionally comprising a droplet collection device, the device comprising a container for holding an immiscible liquid with lower density than water, the container comprising a tip, the tip connectable to the exit of the first microfluidic device and the injection port of the second microfluidic device, the container connectable to a pump, the pump adapted to eject droplets from the tip during injection into the subsequent microfluidic device, optionally wherein the pump is additionally adapted to aspirate droplets into the droplet collection device during collection.

9. Use of the system of any of claims 1-8 for single cell analyses, optionally single cell RNA analyses.

10. A method of single cell RNA analyses, the method comprising:
a) encapsulating a cell or cell structure, and lysis reagent in a microfluidic droplet;
b) incubating the droplet to release the RNA;
c) combining the droplet with: i) a bead; and ii) reverse transcriptase reagent using droplet fusion.

11. The method of claim 10, wherein the method further comprises: a sorting step downstream of encapsulation step a), wherein the sorting step comprises dividing the droplets into a first droplet set and a second droplet set,
optionally wherein in the first droplet set, the droplets comprise live cells; and wherein in the second droplet set, the droplets comprise dead cells, and/or empty droplets, and/or droplets containing more than one cell or cell structure.

12. The method of claims 10-11, implemented using the system of claim 1.

13. A method for single cell analyses, the method comprising:
a) encapsulating a cell or cell structure, lysis reagent and a bead in a microfluidic droplet;
b) incubating the droplets to release the contents of the cell or cell structure; and
c) picoinjecting a reagent into the microfluidic droplet.

14. The method of claims 10-13, wherein step a) results in the following concentrations in the droplet:
a) a protease at a concentration of 1-20 U/ml; and/or
b) a non-ionic detergent at a concentration of 0.1-0.5% v/v.

15. The method of any of claims 13-14, wherein the amount picoinjected in step c) is 0.001 nl-2 nl.

16. The method of any of claims 13-15, wherein the method further comprises a sorting step downstream of encapsulation step a) or piconjection step c), wherein the sorting step comprises sorting the droplets into a first droplet set and a second droplet set,
optionally wherein in the first droplet set, the droplets comprise lysate from live cells; and wherein in the second droplet set, the droplets comprises lysate from dead cells, and/or empty droplets and/or droplets containing more than one cell or cell structure.

17. The method of claims 13-16, wherein the method is implemented using the system of claim 2-5.

18. The method of claim 17, wherein:
a) the microdroplets are collected from the droplet generation microfluidic device with a droplet collection device, the droplet collection device comprising a container, the container comprising an immiscible liquid with lower density than water, optionally a hydrocarbon or silicone oil, the container comprising a tip, wherein the tip is connected to the exit of the droplet generation module to collect droplets into the device;
b) the microfluidic droplets are incubated in the container to lyse the cell; and
c) optionally the droplets are reinjected into the picoinjection device by connecting the container to a pump adapted to eject droplets from the tip.

19. The method of any of claims 13-18 for single cell RNA analyses, wherein the reagent picoinjected at step c) is a reverse transcriptase reagent comprising one or more reverse transcriptase(s) optionally wherein the one or more reverse transcriptase(s) is added to the droplet to result in a concentration of 1-20 kU/ml in the droplet.

20. The method of claim 19, wherein the amount of reverse transcriptase reagent picoinjected is:
a) 0.2-1.5 nl; or
b) 20%-200% the volume of the droplet.

21. The method of claims 10-20, wherein the method includes adding a nucleic acid spike-in as a control.

22. The method of any of claims 10-21, wherein the lysis reagent comprises:
a) a protease, optionally Proteinase K; and/or
b) a non-ionic detergent, optionally IGEPAL CA-630.

23. The method of any of claims 10-22, wherein the bead comprises: a poly-T primer comprising a barcode, and optionally a UMI.

24. The method of any of claims 10-23 wherein the reverse transcriptase reaction comprises a template switching oligonucleotide (TSO), and one or more of the reverse transcriptases has template switching activity.

25. A method of separating nuclear RNA and cytoplasmic RNA for sequencing, the method comprising:
step a) encapsulating a cell and outer membrane lysis reagent in a microfluidic droplet, wherein the outer membrane lysis reagent lyses the outer membrane of the cell;
step b) adding nuclear membrane lysis reagent, wherein the nuclear membrane lysis reagent is for lysing the nuclear membrane; and splitting the droplet into a plurality of droplets to obtain: i) a droplet with nuclear lysate; and ii) a plurality of droplets with cytoplasmic lysate;
step c) adding reverse transcriptase reagent and sorting the droplets into a first droplet comprising nuclear lysate; and a second droplet set comprising cytoplasmic lysate; and
step d) performing reverse transcription of: i) the nuclear RNA and; ii) the cytoplasmic RNA, wherein the cDNA in the cDNA sequencing library comprises a barcode and optionally a UMI.

26. The method of claim 25, wherein:
a) step a) additionally comprises encapsulating a bead; or
b) step c) additionally comprises adding a bead;
wherein the bead comprises a plurality of cDNA synthesis primers adapted to initiate cDNA synthesis; optionally wherein:
c) the bead is a dissolvable bead and removal is via incubation to dissolve the bead; or
d) the bead is removed using the bead extractor device of claims 1-4.

27. The method of claim 25 or 26a), wherein the method is implemented using the system of claim 2 or 6-7,
and wherein:
step a) is implemented with the droplet generation module;
step b) is implemented with the picoinjection module, the picoinjector module optionally comprising the bead extractor device of claims 1-4, and also comprising a droplet splitter downstream from the picoinjector, the droplet splitter adapted to split the droplet into a plurality of smaller droplets, to obtain: i) a droplet with nuclear lysate; and ii) a plurality of droplets with cytoplasmic lysate; and
step c) is implemented with a second picoinjection module, wherein the second picoinjection module further comprises a bifurcated sorting junction downstream of picoinjection, the bifurcated sorting junction in fluid communication with a first exit channel and a second exit channel wherein the bifurcated sorting junction is adapted to divide the droplets into a first droplet set comprising nuclear lysate which exits via the first exit channel and a second droplet set comprising cytoplasmic lysate which exits via the second exit channel.

28. The method of claim 25 or 26b), wherein the method is implemented using the system of claim 2 or 6-7,
and wherein:
step a) is implemented with the droplet generation module;
step b) is implemented with the picoinjection module, the picoinjector module optionally comprising the bead extractor device of claims 1-4, and also comprising a droplet splitter downstream from the picoinjector, the droplet splitter adapted to split the droplet into a plurality of smaller droplets, to obtain: i) a droplet with nuclear lysate; and ii) a plurality of droplets with cytoplasmic lysate; and
step c) is implemented with a droplet fusion module, the droplet fusion module comprising a fusion junction adapted to fuse reverse transcriptase reagent and barcode reagent, optionally a barcoded bead, with the microfluidic droplet.

29. A method of sequencing RNA from a plurality of interacting cells, the method comprising:
step a) encapsulating a first cell and a second cell with a barcoded bead, the bead comprising a plurality of poly-T primers, each primer additionally comprising a first barcode and optionally a UMI;
step b) optionally adding a cell separation reagent;
step c) adding lysis reagent; removal of the bead from the droplet and splitting the droplet into a plurality of droplets to obtain a droplet with lysate from the first cell or cell structure; and a droplet with lysate from the second cell or cell structure.

30. The method of claim 29, additionally comprising:
step d) separating the droplets into two groups: a first group with RNA from the first cell; and a second group with RNA from the second cell and performing reverse transcription on: i) the RNA from the first cell and; ii) the RNA from the second cell, wherein the cDNA in the cDNA sequencing library comprises a barcode and optionally a UMI and the first barcode is the same for the first cell and the second cell.

31. The method of claim 29, additionally comprising:
step d) adding a set of unique second barcodes to the droplet with lysate from the first cell or cell structure and to the droplet with lysate from the second cell or cell structure; and performing reverse transcription on: i) the RNA from the first cell or cell structure and; ii) the RNA from the second cell or cell structure, wherein the cDNA in the cDNA sequencing library comprises a barcode and optionally a UMI and the first barcode is the same for the first cell or cell structure and the second cell or cell structure; and the second barcode is different between the droplet with lysate from the first cell and the lysate from the second cell and distinguishes the RNA from the first cell from the RNA from the second cell.

32. The method of claims 29-31, wherein:
a) the bead is a dissolvable bead and removal is via incubation to dissolve the bead; or
b) the bead is removed using the bead extractor device of claims 38-41.

33. The method of claims 29-32 wherein the method is implemented using the system of claim 2, or 6-7,
and wherein:
step a) is implemented with the droplet generation module, optionally wherein the cell separation reagent is also encapsulated in the droplet by the droplet generation module;
step c) is implemented with the picoinjection module, the picoinjector module optionally comprising the bead extractor device of claims 38-41, and also comprising a droplet splitter downstream from the picoinjector, the droplet splitter adapted to split the droplet into a plurality of smaller droplets, to obtain droplets comprising: i) lysate from the first cell; and ii) lysate from the second cell.

34. The method of claim 30-33 wherein step d) is implemented with:
i) a second picoinjection module for injecting reverse transcriptase; or
ii) a droplet fusion module, the droplet fusion module comprising a fusion junction adapted to fuse reverse transcriptase reagent with the microfluidic droplet, wherein the droplet fusion module comprises a droplet spacer upstream of the fusion junction.

35. The method of claim 34, wherein the second picoinjection module or droplet fusion module additionally comprises a bifurcated sorting junction, the bifurcated sorting junction in fluid communication with a first exit channel and a second exit channel wherein the bifurcated sorting junction is adapted to divide the droplets into a first droplet set comprising lysate from the first cell which exits via the first exit channel and a second droplet set comprising lysate from the second cell which exits via the second exit channel.

36. The method of claim 34 wherein separating the droplets is by fractionation, and each fraction is processed separately during library preparation and wherein a different second barcode is added to each fraction and fused with the first barcode and RNA fragment.

37. The method of claim 34 wherein a set of unique second barcodes is added by droplet fusion, in the droplet fusion module, or by picoinjection in the second picoinjection module and wherein a different second barcode in each droplet is fused with the first barcode and RNA fragment.

38. A bead-extracting microfluidics device for extracting a bead from a microfluidic droplet, the device comprising:
a supply channel, into which microfluidic droplets can be injected;
the supply channel comprising a bifurcated junction in fluid communication with a first exit channel and a second exit channel,
wherein the first exit channel is adapted to extract the bead from the droplet; and the second exit channel is adapted to extract the droplet, the second exit channel having a smaller entrance from the bifurcated junction into the second exit channel than the entrance into the first exit channel from the bifurcated junction; and wherein the second exit channel has a lower flow resistance than the first exit channel.

39. The bead-extracting microfluidics device of claim 38, wherein the lower flow resistance is obtained by any one or more of the following:
a) the second exit channel, downstream of the entrance, has a larger diameter than the first exit channel;
b) the first exit channel is in fluid communication with an auxiliary channel, wherein the auxiliary channel is adapted to flow into the first exit channel to provide higher flow resistance in the first exit channel;
c) the first exit channel has an increased length from the entrance to exit compared to the second exit channel;
d) the second exit channel is attachable to a source of negative pressure.

40. The bead extracting microfluidics device of claims 39a)-c), wherein the angle between the supply channel and the first exit channel is 90.1-179.9 degrees, optionally 120-150 degrees.

41. The bead-extracting microfluidics device of claims 38-40, wherein the diameter of the entrance to the first exit channel is 2-500 microns, optionally 10-100 microns, and the diameter of the entrance to the second exit channel is 10-90%, optionally 40-60% the width of the entrance to the first exit channel.

42. The bead extracting microfluidics device of any of claims 38-41, wherein the device further comprises a droplet splitter downstream from the second exit channel, the droplet splitter adapted to split the extracted droplet into a plurality of smaller droplets.

43. The bead extracting microfluidics device of claim 42, wherein the droplet splitter comprises a plurality of T-junctions; a single flow-focusing junction, or a step emulsification geometry.

44. A method of extracting a bead from a microfluidic droplet, the method comprising:
a) providing a microfluidic droplet, the droplet comprising a bead; and
b) extracting the bead from the microfluidic droplet to provide: i) the bead; and ii) a droplet which does not comprise the bead.

45. The method of claim 44, wherein the method is for analyses of single cells, optionally wherein the microfluidic droplet comprises cell lysate.

46. The method of claims 44-45, wherein the bead comprises a first analyte, optionally captured on a first capture agent.

47. The method of claim 45, wherein the bead comprises a plurality of capture agents, each capture agent adapted to bind a different cell analyte, wherein one or more of the capture agents has a different cleavable linker.

48. The method of claim 47, wherein the plurality of capture agents have the same barcode.

49. The method of claims 47-48, comprising:
a) cleaving a first capture agent and releasing a first analyte from the bead;
b) extracting the bead from the microfluidic droplet to provide: i) the bead; and ii) a first droplet which does not comprise the bead, the droplet comprising the first analyte; and optionally,
c) cleaving a second capture agent and releasing a second analyte from the bead; and
d) extracting the bead from the microfluidic droplet to provide: i) the bead; and ii) a second droplet which does not comprise the bead, the droplet comprising the second analyte.

50. The method of claims 44-47, wherein one or more analyte is immobilized within the bead.

51. The method of any of claims 44-50, wherein the method is implemented using the bead extracting microfluidics device of claims 38-43, the method comprising:
a) injecting a microfluidic droplet comprising a bead into the supply channel of the device of any one of claims 38-43; and
b) flowing the microfluidic droplet into the bifurcated sorting junction to split the droplet into: a bead which flows into the first exit channel; and a droplet which does not contain the bead which flows into the second exit channel.

52. Use of the bead-extracting microfluidics device of claims 38-43 for splitting a microfluidic droplet, optionally for single cell analyses.

53. A bead and one or more microfluidic droplets, wherein the bead and microfluidic droplet(s) comprise different analytes from a single cell, optionally wherein the analytes on the bead and the droplets comprise the same barcode.
